(19) United States
(12) Patent Application Publication
(10) Pub. No.: US 2023/0271943 A1
(43) Pub. Date: Aug. 31, 2023

(54) COMPOUNDS AS DHODH INHIBITORS

(71) Applicant: Nanjing Zenshine Pharmaceuticals Co., Ltd., Nanjing (CN)

(72) Inventor: Xiaolin Hao, Foster City, CA (US)

(21) Appl. No.: 18/005,357

(22) PCT Filed: Jul. 13, 2021
(86) PCT No.: PCT/CN2021/106082

§ 371 Date: Jan. 13, 2023

Related U.S. Application Data
(60) Provisional application No. 63/051,410, filed on Jul. 14, 2020.

(51) Int. Cl. C07D 403/04; C07D 401/04

(52) U.S. Cl. CPC C07D 403/04; C07D 401/04

(57) ABSTRACT

Provided are 5-oxo-4,5-dihydro-1H-1,2,4-triazole compounds, which are the inhibitors of dihydroorotate dehydrogenase (DHODH). More specifically, provided are the preparation of the compounds and their use in the preparation of pharmaceutical composition for the treatment of various diseases, conditions and disorders related to DHODH activity.

### FIELD OF THE INVENTION

This invention relates generally to compounds as inhibitors of dihydroorotate dehydrogenase (DHODH) activity. More specifically, the invention further relates to the preparation of the compounds and their use in pharmaceutical compositions for the treatment of various diseases, conditions and disorders related to DHODH activity.

### BACKGROUND

Pyrimidines are required for the biosynthesis of bio-macromolecules including DNA, RNA, glycoproteins, and phospholipids, and are linked by phosphodiester bridges to purine nucleotides in double-stranded DNA, both in nucleus and mitochondria (Loeffler, M. et al., Encyc. Biol. Chem., 2004, 3, 600-605.) There are two ways for the synthesis of pyrimidines in most of the organisms: salvage pathway and de novo synthesis from small metabolites. For normal differentiated cells, salvage pathway is the main source of pyrimidine nucleotides. In order to meet increased demand for nucleic acid precursors and other cellular components, activated T cell and other rapidly proliferating cells depend heavily on de novo synthesis. (Fairbanks, L.D.et, al., J. Biol. Chem., 1995, 270, 29682-29689.)

Dihydroorotate dehydrogenase (DHODH) is a flavin-dependent enzyme located in the mitochondria and is responsible for the 4th novo pyrimidine synthesis. DHODH catalyzes conversion of DHO to ORO, which represents the rate limiting step in de novo pyrimidine biosynthesis (Loffler M. et al., Mol Cell Biochem. 1997,174, 125-129). DHODH also catalyzes the reduction of flavin mononucleotide (FMN) to dihydroflavin mononucleotide (FMNH2), which comprises two half reactions of redox couple. The co-substrate electron acceptor used by DHODH varies in different organisms. In human DHODH, flavin cofactor is FMN and ubiquinone (CoQ) is the second substrate. DHODH from higher eukaryotes exhibits a two-site ping-pong mechanism with FMN serving as an intermediate in the electron transfer. (Hansen, M., et al., Protein Sci., 2004, 13, 1031-1042. Hurt, D.E. et al., Acta Crystallogr D Biol Crystallogr. 2006, 62, 312-323.)

As DHODH is involved and regarded as a central enzyme in the process of pyrimidine’s biosynthesis and mitochondria electron transfer chain, inhibition of DHODH lowers intracellular pyrimidine nucleotides pools in cells. Reduced levels of pyrimidine nucleotides by DHODH inhibition halt abnormal cell proliferation, since rapid cell proliferation often depends on de novo synthesis of pyrimidine nucleotides, which makes DHODH as an attractive target for biological and clinical applications for the treatment of cancer, arthritis and malaria.

Inhibitors of DHODH have proven efficacy for the treatment of many diseases including bacterial and viral infections, parasitic diseases (i.e., malaria), autoimmune diseases and cancer (Reis, R, et al., Arch.Biochem.Biophys.2017, 632, 175-191). Inhibition of DHODH depletes intracellular pyrimidine nucleotide pools and results in cell cycle arrest in S-phase, sensitization to current chemotherapies, and differentiation in neural crest cells and acute myeloid leukemia (AML) (Koundinya, M., et al., Cell Chem Biol, 2018, 25, 705-717). Therefore, DHODH inhibitors have the potential as mono therapy or part of combination therapies for the treatment of cancer.

DHODH’s relevance in cancer was recognized nearly six decades ago when Smith et al. noted elevated DHODH activity in leukemic cells (Smith L.H., et al., Blood, 1960, 15, 360-369;), Recent reports have revisited the link between DHODH inhibition and antiproliferative effects in cells, it was found that DHODH inhibition correlates with decreased cell growth in most cancer cell lines (Aguirre A.J., et al., Cancer Discov, 2016, 6, 914-929),, such as HCT-116 from colorectal carcinoma, Jurkat from leukemia, and HUT-78 from lymphoma.

In addition to application in the treatment of cancer, DHODH inhibitors also show broad-spectrum antiviral activity, which is mainly attributed to the depletion of the nucleosides necessary for replication of the viral genome (Hoffmann H.H., et al., Proc Natl Acad Sci USA 2011, 108, 15366-15371; Wachsman M, et al., Antivir Chem Chemother 1996, 40, 434-466; Chenung N.N., et al., J Gen Virol. 2017, 98, 946-954). Several independent studies searching for host-targeting antiviral (HTA) drugs collectively end up with compounds targeting the host’s pyrimidine synthesis pathway to inhibit virus infections, which indicates that the replication of viruses is widely dependent on the host pyrimidine synthesis (Chung, D. H., et al., Antimicrob. Agents Chemother. 2016, 60, 4552-4562). Cmp1 and FK778 targeting DHODH have been confirmed to inhibit the DNA virus (CMV) replication in RAG-/- mice (Marschall, M., et al., Antiviral Res. 2013, 100, 640-648. Zeng, H., et al., Transplantation 2005, 79, 17-22). Recently, it was found that two representative inhibitors of DHODH, S312 and S416 show broad-spectrum antiviral effects against various RNA virus, including influenza A virus, Zika virus, Ebola virus, and SARS-CoV-2 (Xiong R. et al., BioRxiv preprint 2020, https://doi.org/10.1101/2020.03.11.983056).

First generation DHODH inhibitors such as leflunomide were developed as an immune-modulatory agents for the treatment of the symptoms of rheumatoid arthritis. However, the weak activity and non-specificity of early DHODH inhibitors were suspected to be the cause of lack of efficacy in human and clinical set-backs. Recently, more evidence led to renewed interest in DHODH mechanism study and clinical use. Therefore there is an essential need for developing a generation of new inhibitors of DHODH.

### SUMMARY OF THE INVENTION

Compounds and pharmaceutically acceptable salts, stereoisomers, prodrugs, or solvates thereof useful for inhibiting DHODH are described herein. Compositions including the compounds are also provided, as are methods of using and making the compounds. The compound provided herein can be used in treating diseases, conditions or disorders that are mediated by DHODH.

In the first aspect, provided is a compound of Formula (I):

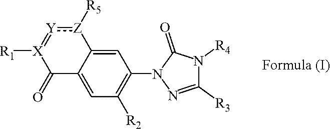

or a pharmaceutically acceptable salt, stereoisomer, prodrug, or solvate thereof, wherein:
X and Z are independently N or C;
Y is selected from the group consisting of N and CR₇;
wherein when X is N, Z is C, 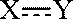
is a single bond, 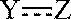
is a double bond;
when X is C, Z is N, 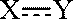
is a double bond, 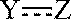
is a single bond;
R₁ is selected from the group consisting of
   C₁-C₈ alkyl;
   C₂-C₈ haloalkyl;
   C₃-C₈ cycloalkyl which is optionally substituted with 1-6 halogen atoms or a group selected from hydroxyl and phenyl, wherein said phenyl substituent is optionally substituted with 1-4 halogen atoms or a group selected from C₁-C₃ alkyl, C₁-C₄ haloalkyl, C₁-C₃ alkoxy, CN and hydroxyl;
   C₃-C₆ alkyl which is substituted with a monocyclic- or bicyclic heteroaryl group;
   (C₂-C₆ hydroxyalkyl)-O-(C₂-C₆ alky);
   (C₃-C₆ alkyl)-N(R₇)(R₈);
   (C₃-C₈ cycloalkyl)-N(R₇)(R₈);
   (C₃-C₆-alkyl)—C(═O)N(R₇)(R₈);
   4-7-membered heterocycloalkyl, wherein said 4-7-membered heterocycloalkyl is optionally substituted with one or two substituents which is independently selected from the group consisting of C₁-C₃ alkyl, 5- to 6-membered heteroaryl, —C(═O)O(C₁-C₄ alkyl), —C(═O)(C₁-C₃-alkyl), —C(═O)(C₃-C₆-cycloalkyl), —S(═O)₂(C₁-C₆-alkyl) and oxo (═O);
   a phenyl which is optionally substituted with one, two, three, four or five substituents, wherein each substituent is independently selected from the group consisting of halogen, C₁-C₆ alkyl, C₃-C₈ cycloalkyl, C₁-C₆ haloalkyl, C₂-C₆-alkenyl, C₂-C₆ alkynyl, aryl, -(C₁-C₆ alkyl)-aryl, -aryl-(C₁-C₆ alkyl), hydroxyl, cyano, C₁-C₆ hydroxyalkyl, C₁-C₄ alkoxy, -O(C₂-C₆ alkenyl), C₁-C₆ haloalkoxy, C₃-C₈ cycloalkoxy, aryl, —O—aryl, cyano, —C(═O)OR₆, —C(═O)N(R₇)(R₈), —N(R₇)(R₈), -(C₁-C₆ alkyl)-N(R₇)(R₈), -(C₁-C₆ alkyl)—C(═O)OR₆, -(C₁-C₆ alkyl)—C(═O)N(R₇)(R₈), —O—C(═O)—(C₁-C₆ alkyl), —SH, —S—(C₁-C₆ alkyl), —S—(C₂-C₆ alkenyl), —S(═O)₂N(R₇)(R₈), —S(═O)₂(C₁-C₆ alkyl), —S(═O)₂—(C₂-C₆ alkenyl), —S(═O)(═NR₁₁)(C₁-C₃ alkyl), —N(O)₂, —P(═O)(C₁-C₃ alkyl)₂;
   bicyclic aryl and 5-10 membered heteroaryl, wherein said bicyclic aryl and said 5-10 membered heteroaryl are optionally substituted with one, two or three substituents, wherein each substituent is independently halogen atom or a group selected from C₁-C₆ alkyl, C₃-C₈ cycloalkyl, C₁-C₆ haloalkyl, C₂-C₆ alkenyl, C₂-C₆ alkynyl, aryl, -(C₁-C₆ alkyl)-aryl, -aryl-(C₁-C₆ alkyl), hydroxy, cyano, C₁-C₆ hydroxyalkyl, C₁-C₄-alkoxy, -O(C₂-C₆ alkenyl), C₁-C₆-haloalkoxy, C₃-C₈-cycloalkoxy, aryl, —O—aryl, cyano, —C(═O)OR₆, —C(═O)N(R₇)(R₈), —N(R₇)(R₈), -(C₁-C₆-alkyl)-N(R₇)(R₈), -(C₁-C₆-alkyl)—C(═O)OR₆, -(C₁-C₆-alkyl)—C(═O)N(R₇)(R₈), —O—C(═O)—(C₁-C₆-alkyl), —S—(C₁-C₆ alkyl), —S—(C₂-C₆-alkenyl), —S(═O)₂N(R₇)(R₈), —S(═O)₂(C₁-C₆-alkyl), —S(═O)₂—(C₂-C₆ alkenyl), —S(═O)(═NR₁₁)(C₁-C₃alkyl), —N(O)₂, —P(═O)(C₁-C₃-alkyl)₂;
R₂ is selected from the group consisting of hydrogen, D, CN and halogen;
R₃ is selected from the group consisting of halogen, C₁-C₆ alkyl, C₃-C₈ cycloalkyl, C₁-C₆ haloalkyl, C₁-C₆ hydroxyalkyl, (C₁-C₃ alkoxy)-(C₁-C₆ alkyl), C₂-C₆ alkenyl, C₂-C₆ alkynyl, C₁-C₆ alkoxy, C₁-C₆ alkylsulfanyl, (C₁-C₆ alkyl)-N(R₇)(R₈), —N(R₇)(R₈), —C(═O)OR₆, —C(═O)N(R₇)(R₈), and S(═O)(═NR₁₁)(C₁-C₃ alkyl);
R₄ is hydrogen or selected from the group consisting of C₁-C₆ alkyl, C₂-C₆ alkylenyl, C₃-C₈ cycloalkyl, C₂-C₆ haloalkyl, C₂-C₆ hydroxyalkyl and (C₂-C₆ alkyl)-N(R₇)(R₈), wherein said C₁-C₆ alkyl is optionally substituted with a group selected from C₃-C₈ cycloalkyl and phenyl, wherein said phenyl is optionally substituted with one, two or three substituents, with each substituent independently selected from the group consisting of halogen, C₁-C₃ alkyl, C₁-C₄ haloalkyl, C₁-C₃ alkoxy and hydroxyl;
R₅ is selected from the group consisting of C₁-C₆ alkyl, C₃-C₈ cycloalkyl, C₁-C₆ haloalkyl, C₁-C₆ hydroxyalkyl, C₂-C₆ alkenyl, C₂-C₆ alkynyl, C₄-C₈ cycloalkenyl, (C₁-C₆ alkyl)-N(R₇)(R₈), -(C₁-C₆ alkyl)-(4- to 7-membered nitrogen containing heterocycloalkyl) and phenyl, wherein said C₁-C₆ alkyl is optionally substituted with C₃-C₈ cycloalkyl or NR₇R₈, said 4- to 7-membered nitrogen containing heterocycloalkyl is optionally substituted with a C₁-C₃ alkyl which is connected to the 4- to 7-membered nitrogen containing heterocycloalkyl via carbon atom of the heterocycloalkyl, and said phenyl is substituted with 1-4 halogen atoms or C₁-C₃ alkyl, C₁-C₄ haloalkyl, C₁-C₃ alkoxy and hydroxyl;
R₆ is hydrogen or selected from the group consisting of C₁-C₆ alkyl and benzyl;
R₇ and R₈ are independently hydrogen or selected from the group consisting of C₁-C₆ alkyl, C₂-C₆ hydroxyalkyl, (C₂-C₆ alkyl)-N(R₉)(R₁₀), and C₃-C₆ cycloalkyl, or R₇ and R₈ together with the nitrogen to which they are attached represent a nitrogen containing 4- to 7-membered heterocycloalkyl, wherein said 4- to 7-membered nitrogen containing heterocycloalkyl is optionally substituted with C₁-C₃ alkyl, —S(═O)₂(C₁-C₃ alkyl) and —C(═O)O(C₁-C₄ alkyl);
R₉ and R₁₀ are independently hydrogen or C₁-C₃ alkyl, or R₉ and R₁₀ together with the nitrogen to which they are attached represent a nitrogen containing 4- to 7-membered heterocycloalkyl group;
R₁₁ is hydrogen, cyano or C(=O)(C₁-C₃ haloalkyl).

In some embodiments, provided is the compound of Formula (I), or a pharmaceutically acceptable salt, stereoisomer, prodrug, or solvate thereof, wherein Y is N, X is C, Z is N,

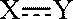

is a double bond,

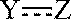

is a single bond.

In the second and preferable aspect, provided is a compound of Formula (II):

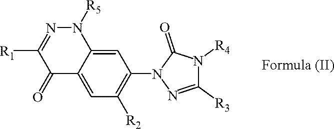

or a pharmaceutically acceptable salt, stereoisomer, prodrug, or solvate thereof, wherein R₁, R₂, R₃, R₄ and R₅ are disclosed herein as Formula (I).

In another aspect, provided is a method for treating or preventing DHODH-mediated diseases, conditions or disorders, comprising administering to a subject in need a therapeutically effective amount of the compound of Formula (I) or Formula (II), or a pharmaceutically acceptable salt, stereoisomer or solvate thereof.

Also provided is a pharmaceutical composition comprising: (i) the compound of Formula (I) or Formula(II), or a pharmaceutically acceptable salt, stereoisomer or solvate thereof; and (ii) one or more pharmaceutically acceptable carriers or excipients. Kits comprising the compound of Formula (I) or Formula (II), or a pharmaceutically acceptable salt, stereoisomer or solvate thereof and optionally instructions for use are also provided.

Also provided is a use of the compound of Formula (I) or Formula (II), or a pharmaceutically acceptable salt, stereoisomer or solvate thereof as a medicament. Compounds as detailed herein or a pharmaceutically acceptable salt, stereoisomer or solvate thereof are also provided for the manufacture of a medicament for the treatment or prevention of DHODH-mediated diseases, conditions or disorders, such as viral infection, cancer, inflammatory disorders and so on.

### DETAILED DESCRIPTION

### Definitions

To facilitate understanding by those skilled in the art, some terms in the invention are described. These descriptions should not be regarded as limitation of the protection scope of the present invention.

For use herein, unless clearly indicated otherwise, use of the terms “a”, “an” and the like refers to one or more.

“A pharmaceutically acceptable salt” or “pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases which can be used to prepared salts include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc = “directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of an individual.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In some embodiments, an effective amount is an amount sufficient to delay development. In some embodiments, an effective amount is an amount sufficient to prevent or delay recurrence. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “subject” is a mammal, including humans. A subject includes, but is not limited to, human, bovine, horse, feline, canine, rodent, or primate. In some embodiments, the subject is human.

The term “alkyl”, by itself or as part of another group, refers to a hydrocarbon group selected from linear and branched, saturated hydrocarbon groups comprising, for example, 1, 2, 3, 4, 5, 6, 7 or 8, carbon atoms. Examples of alkyl groups include without limitation to methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkenyl”, by itself or as part of another group, refers to a hydrocarbon group selected from linear and branched hydrocarbon groups comprising at least one C═C double bond and, for example, 2, 3, 4, 5, 6, 7 or 8, carbon atoms. Examples of alkenyl groups include without limitation to ethenyl, prop-1-enyl, prop-2-enyl, 2-methylprop-1-enyl, but-1-enyl, but-2-enyl, but-3-enyl, but-1,3-dienyl, 2-methylbut-1,3-dienyl, hex-1-enyl, hex-2-enyl, hex-3-enyl, hex-4-enyl, and hexa-1,3-dienyl.

The term “alkynyl”, by itself or as part of another group, refers to a hydrocarbon group selected from linear and branched hydrocarbon group, comprising at least one C═C triple bond and, for example, 2, 3, 4, 5, 6, 7 or 8, carbon atoms. Examples of alkynyl groups include without limitation to ethynyl, 1-propynyl, 2-propynyl (propargyl), 1-butynyl, 2-butynyl, and 3-butynyl.

The term “cycloalkyl”, by itself or as part of another group, refers to cyclic saturated hydrocarbon groups, including C₃, C₄, C₅, C₆, C₇, and C₈ cycloalkyl groups. Examples of cycloalkyl groups include without limitation to cyclopropyl, cyclobutyl, cyclopentyl, and cyclohexyl.

The term “heterocycloalkyl”, by itself or as part of another group, refers to 4-, 5-, 6- or 7-membered monocyclic saturated ring structure containing one, two or three heteroatom selected from the group consisting of O, N and S. Examples of heterocycloalkyl groups include without limitation to oxetanyl, tetrahydrofuranyl, tetrahydropyranyl, oxepanyl, azetidinyl, pyrrolidinyl, piperidinyl, piperazinyl, azepanyl, morpholinyl and oxazepanyl.

The term “aryl”, by itself or as part of another group, refers to monocyclic and bicyclic aromatic groups containing 6-10 carbons in the ring portion (such as the monocyclic aryl phenyl or the bicyclic aryl naphthyl including 1-naphthyl and 2-naphthyl).

The term “heteroaryl”, by itself or as part of another group, refers to aromatic 5- or 6-membered monocyclic groups and 9- or 10-membered bicyclic groups, which have one, two or three heteroatom selected from the group consisting of O, N and S. Exemplary monocyclic heteroaryl groups include pyrrolyl, pyrazolyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and pyranyl. Exemplary bicyclic heteroaryl groups include indolyl, benzothiazolyl, benzodioxolyl, benzoxazolyl, benzothienyl, quinolinyl, isoquinolinyl, benzimidazolyl, benzopyranyl, benzofuranyl, cinnolinyl, quinoxalinyl and indazolyl.

The term “halogen” or “halo”, by itself or as part of another group, refers to fluoro (F), chloro (Cl), bromo (Br) and iodo (I).

It is understood that aspects and variations described herein also include “consisting” and/or “consisting essentially of” aspects and variations.

### Compounds and Pharmaceutical Compositions

In the first aspect, provided is a compound of the Formula (I):

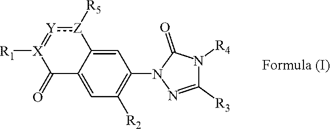

or a pharmaceutically acceptable salt, stereoisomer, prodrug, or solvate thereof, wherein:
X and Z are independently N or C;
Y is selected from the group consisting of N and CR₇;
   wherein when X is N, Z is C, 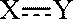
   is a single bond, 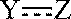
   is a double bond;
   when X is C, Z is N, 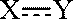
   is double bond, 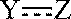
   is a single bond;
R₁ is selected from the group consisting of
   C₁-C₈ alkyl;
   C₂-C₈ haloalkyl;
   C₃-C₈ cycloalkyl which is optionally substituted with 1-6 halogen atoms or a group selected from hydroxyl and phenyl, wherein said phenyl substituent is optionally substituted with 1-4 halogen atoms or a group selected from C₁-C₃ alkyl, C₁-C₄ haloalkyl, C₁-C₃ alkoxy, CN and hydroxyl;
   C₃-C₆ alkyl which is substituted with a monocyclic- or bicyclic heteroaryl group;
   (C₂-C₆ hydroxyalkyl)-O-(C₂-C₆ alky);
   (C₃-C₆ alkyl)-N(R₇)(R₈);
   (C₃-C₈ cycloalkyl)-N(R₇)(R₈);
   (C₃-C₆-alkyl)—C(═O)N(R₇)(R₈);
   4-7-membered heterocycloalkyl, wherein said 4-7-membered heterocycloalkyl is optionally substituted with one or two substituents which is independently selected from the group consisting of C₁-C₃ alkyl, 5- to 6-membered heteroaryl, —C(═O)O(C₁-C₄ alkyl), —C(═O)(C₁-C₃-alkyl), —C(═O)(C₃-C₆-cycloalkyl), —S(═O)₂(C₁-C₆-alkyl) and oxo (═O);
   a phenyl which is optionally substituted with one, two, three, four or five substituents, wherein each substituent is independently selected from the group consisting of halogen, C₁-C₆ alkyl, C₃-C₈ cycloalkyl, C₁-C₆ haloalkyl, C₂-C₆-alkenyl, C₂-C₆ alkynyl, aryl, -(C₁-C₆ alkyl)-aryl, -aryl-(C₁-C₆ alkyl), hydroxyl, cyano, C₁-C₆ hydroxyalkyl, C₁-C₄ alkoxy, -O(C₂-C₆ alkenyl), C₁-C₆ haloalkoxy, C₃-C₈ cycloalkoxy, aryl, —O—aryl, cyano, —C(═O)OR₆, —C(═O)N(R₇)(R₈), —N(R₇)(R₈), -(C₁-C₆ alkyl)-N(R₇)(R₈), -(C₁-C₆-alkyl)—C(═O)OR₆, -(C₁-C₆ alkyl)—C(═O)N(R₇)(R₈), —O—C(═O)—(C₁-C₆ alkyl), —SH, —S—(C₁-C₆ alkyl), —S—(C₂-C₆ alkenyl), —S(═O)₂N(R₇)(R₈), —S(═O)₂(C₁-C₆ alkyl), —S(═O)₂—(C₂-C₆ alkenyl), —S(═O)(═NR₁₁)(C₁-C₃ alkyl), —N(O)₂, —P(═O)(C₁-C₃ alkyl)₂;
   bicyclic aryl and 5-10 membered heteroaryl, wherein said bicyclic aryl and said 5-10 membered heteroaryl are optionally substituted with one, two or three substituents, wherein each substituent is independently halogen atom or a group selected from C₁-C₆ alkyl, C₃-C₈ cycloalkyl, C₁-C₆ haloalkyl, C₂-C₆ alkenyl, C₂-C₆ alkynyl, aryl, -(C₁-C₆ alkyl)-aryl, -aryl-(C₁-C₆ alkyl), hydroxy, cyano, C₁-C₆ hydroxyalkyl, C₁-C₄ alkoxy, -O(C₂-C₆ alkenyl), C₁-C₆ haloalkoxy, C₃-C₈ cycloalkoxy, aryl, —O—aryl, cyano, —C(═O)OR₆, —C(═O)N(R₇)(R₈), —N(R₇)(R₈), -(C₁-C₆ alkyl)-N(R₇)(R₈), -(C₁-C₆ alkyl)—C(═O)OR₆, -(C₁-C₆ alkyl)—C(═O)N(R₇)(R₈), —O—C(═O)—(C₁-C₆ alkyl), —S—(C₁-C₆ alkyl), —S—(C₂-C₆ alkenyl), —S(═O)₂N(R₇)(R₈), —S(═O)₂(C₁-C₆ alkyl), —S(═O)₂—(C₂-C₆ alkenyl), —S(═O)(═NR₁₁)(C₁-C₃ alkyl), —N(O)₂, —P(═O)(C₁-C₃ alkyl)₂;
R₂ is selected from the group consisting of hydrogen, D, CN and halogen;
R₃ is selected from the group consisting of halogen, C₁-C₆ alkyl, C₃-C₈ cycloalkyl, C₁-C₆ haloalkyl, C₁-C₆ hydroxyalkyl, (C₁-C₃ alkoxy)-(C₁-C₆ alkyl), C₂-C₆ alkenyl, C₂-C₆ alkynyl, C₁-C₆ alkoxy, C₁-C₆ alkylsulfanyl, (C₁-C₆ alkyl)-N(R₇)(R₈), —N(R₇)(R₈), —C(═O)OR₆, —C(═O)N(R₇)(R₈), and S(═O)(═NR₁₁)(C₁-C₃ alkyl);
R₄ is hydrogen or selected from the group consisting of C₁-C₆ alkyl, C₂-C₆ alkylenyl, C₃-C₈ cycloalkyl, C₂-C₆ haloalkyl, C₂-C₆ hydroxyalkyl and (C₂-C₆ alkyl)-N(R₇)(R₈), wherein said C₁-C₆ alkyl is optionally substituted with a group selected from C₃-C₈ cycloalkyl and phenyl, wherein said phenyl is optionally substituted with one, two or three substituents, with each substituent independently selected from the group consisting of halogen, C₁-C₃ alkyl, C₁-C₄ haloalkyl, C₁-C₃ alkoxy and hydroxyl;
R₅ is selected from the group consisting of C₁-C₆ alkyl, C₃-C₈ cycloalkyl, C₁-C₆ haloalkyl, C₁-C₆ hydroxyalkyl, C₂-C₆ alkenyl, C₂-C₆ alkynyl, C₄-C₈ cycloalkenyl, (C₁-C₆ alkyl)-N(R₇)(R₈), -(C₁-C₆ alkyl)-(4- to 7-membered nitrogen containing heterocycloalkyl) and phenyl, wherein said C₁-C₆ alkyl is optionally substituted with C₃-C₈ cycloalkyl or NR₇R₈, said 4- to 7-membered nitrogen containing heterocycloalkyl is optionally substituted with a C₁-C₃ alkyl which is connected to the 4- to 7-membered nitrogen containing heterocycloalkyl via carbon atom of the heterocycloalkyl, and said phenyl is substituted with one, two, three or four substituents, and each substituent is selected from the group consisting of halogen, C₁-C₃ alkyl, C₁-C₄ haloalkyl, C₁-C₃ alkoxy and hydroxyl;
R₆ is hydrogen or selected from the group consisting of C₁-C₆ alkyl and benzyl;
R₇ and R₈ are independently hydrogen or selected from the group consisting of C₁-C₆ alkyl, C₂-C₆ hydroxyalkyl, (C₂-C₆ alkyl)-N(R₉)(R₁₀), and C₃-C₆ cycloalkyl, or R₇ and R₈ together with the nitrogen to which they are attached represent a nitrogen containing 4- to 7-membered heterocycloalkyl, wherein said 4- to 7-membered nitrogen containing heterocycloalkyl is optionally substituted with C₁-C₃ alkyl, —S(═O)₂(C₁-C₃ alkyl) and —C(═O)O(C₁-C₄ alkyl);
R₉ and R₁₀ are independently hydrogen or C₁-C₃ alkyl, or R₉ and R₁₀ together with the nitrogen to which they are attached represent a nitrogen containing 4- to 7-membered heterocycloalkyl group;
R₁₁ is hydrogen, cyano or C(=O)(C₁-C₃ haloalkyl).

In the second and preferable aspect, provided is a compound of Formula (II):

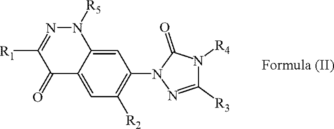

or a pharmaceutically acceptable salt, stereoisomer, prodrug, or solvate thereof, wherein:
R₁ is selected from the group consisting of
   C₁-C₈ alkyl;
   C₂-C₈ haloalkyl;
   C₃-C₈ cycloalkyl which is optionally substituted with 1-6 halogen atoms or a group selected from hydroxyl and phenyl, wherein said phenyl substituent is optionally substituted with 1-4 halogen atoms or a group selected from C₁-C₃ alkyl, C₁-C₄ haloalkyl, C₁-C₃ alkoxy, CN and hydroxyl;
   C₃-C₆ alkyl which is substituted with a monocyclic- or bicyclic heteroaryl group;
   (C₂-C₆ hydroxyalkyl)-O-(C₂-C₆ alky);
   (C₃-C₆ alkyl)-N(R₇)(R₈);
   (C₃-C₈ cycloalkyl)-N(R₇)(R₈);
   (C₃-C₆-alkyl)—C(═O)N(R₇)(R₈);
   4-7-membered heterocycloalkyl, wherein said 4-7-membered heterocycloalkyl is optionally substituted with one or two substituents which is independently selected from the group consisting of C₁-C₃ alkyl, 5- to 6-membered heteroaryl, —C(═O)O(C₁-C₄ alkyl), —C(═O)(C₁-C₃-alkyl), —C(═O)(C₃-C₆-cycloalkyl), —S(═O)₂(C₁-C₆-alkyl) and oxo (═O);
   a phenyl which is optionally substituted with one, two, three, four or five substituents, wherein each substituent is independently selected from the group consisting of halogen, C₁-C₆ alkyl, C₃-C₈ cycloalkyl, C₁-C₆ haloalkyl, C₂-C₆-alkenyl, C₂-C₆ alkynyl, aryl, -(C₁-C₆ alkyl)-aryl, -aryl-(C₁-C₆ alkyl), hydroxyl, cyano, C₁-C₆ hydroxyalkyl, C₁-C₄ alkoxy, -O(C₂-C₆ alkenyl), C₁-C₆ haloalkoxy, C₃-C₈ cycloalkoxy, aryl, —O—aryl, cyano, —C(═O)OR₆, —C(═O)N(R₇)(R₈), —N(R₇)(R₈), -(C₁-C₆ alkyl)-N(R₇)(R₈), -(C₁-C₆ alkyl)—C(═O)OR₆, -(C₁-C₆ alkyl)—C(═O)N(R₇)(R₈), —O—C(═O)—(C₁-C₆ alkyl), —SH, —S—(C₁-C₆ alkyl), —S—(C₂-C₆ alkenyl), —S(═O)₂N(R₇)(R₈), —S(═O)₂(C₁-C₆ alkyl), —S(═O)₂—(C₂-C₆ alkenyl), —S(═O)(═NR₁₁)(C₁-C₃ alkyl), —N(O)₂, —P(═O)(C₁-C₃ alkyl)₂;
   bicyclic aryl and 5-10 membered heteroaryl, wherein said bicyclic aryl and said 5-10 membered heteroaryl are optionally substituted with one, two or three substituents, wherein each substituent is independently halogen atom or a group selected from C₁-C₆ alkyl, C₃-C₈ cycloalkyl, C₁-C₆ haloalkyl, C₂-C₆ alkenyl, C₂-C₆ alkynyl, aryl, -(C₁-C₆ alkyl)-aryl, -aryl-(C₁-C₆ alkyl), hydroxy, cyano, C₁-C₆ hydroxyalkyl, C₁-C₄-alkoxy, -O(C₂-C₆ alkenyl), C₁-C₆-haloalkoxy, C₃-C₈-cycloalkoxy, aryl, —O—aryl, cyano, —C(═O)OR₆, —C(═O)N(R₇)(R₈), —N(R₇)(R₈), -(C₁-C₆-alkyl)-N(R₇)(R₈), -(C₁-C₆-alkyl)—C(═O)OR₆, -(C₁-C₆-alkyl)—C(═O)N(R₇)(R₈), —O—C(═O)—(C₁-C₆-alkyl), —S—(C₁-C₆ alkyl), —S—(C₂-C₆-alkenyl), —S(═O)₂N(R₇)(R₈), —S(═O)₂(C₁-C₆-alkyl), —S(═O)₂—(C₂-C₆ alkenyl), —S(═O)(═NR₁₁)(C₁-C₃alkyl), —N(O)₂, —P(═O)(C₁-C₃-alkyl)₂;
R₂ is selected from the group consisting of hydrogen, D, CN and halogen;
R₃ is selected from the group consisting of halogen, C₁-C₆ alkyl, C₃-C₈ cycloalkyl, C₁-C₆ haloalkyl, C₁-C₆ hydroxyalkyl, (C₁-C₃ alkoxy)-(C₁-C₆ alkyl), C₂-C₆ alkenyl, C₂-C₆ alkynyl, C₁-C₆ alkoxy, C₁-C₆ alkylsulfanyl, (C₁-C₆ alkyl)-N(R₇)(R₈), —N(R₇)(R₈), —C(═O)OR₆, —C(═O)N(R₇)(R₈), and S(═O)(═NR₁₁)(C₁-C₃ alkyl);
R₄ is hydrogen or selected from the group consisting of C₁-C₆ alkyl, C₂-C₆ alkylenyl, C₃-C₈ cycloalkyl, C₂-C₆ haloalkyl, C₂-C₆ hydroxyalkyl and (C₂-C₆ alkyl)-N(R₇)(R₈), wherein said C₁-C₆ alkyl is optionally substituted with a group selected from C₃-C₈ cycloalkyl and phenyl, wherein said phenyl is optionally substituted with one, two or three substituents, with each substituent independently selected from the group consisting of halogen, C₁-C₃ alkyl, C₁-C₄ haloalkyl, C₁-C₃ alkoxy and hydroxyl;
R₅ is selected from the group consisting of C₁-C₆ alkyl, C₃-C₈ cycloalkyl, C₁-C₆ haloalkyl, C₁-C₆ hydroxyalkyl, C₂-C₆ alkenyl, C₂-C₆ alkynyl, C₄-C₈ cycloalkenyl, (C₁-C₆ alkyl)-N(R₇)(R₈), -(C₁-C₆ alkyl)-(4- to 7-membered nitrogen containing heterocycloalkyl) and phenyl, wherein said C₁-C₆ alkyl is optionally substituted with C₃-C₈ cycloalkyl or NR₇R₈, said 4- to 7-membered nitrogen containing heterocycloalkyl is optionally substituted with a C₁-C₃ alkyl which is connected to the 4- to 7-membered nitrogen containing heterocycloalkyl via carbon atom of the heterocycloalkyl, and said phenyl is substituted with 1-4 halogen atoms or C₁-C₃ alkyl, C₁-C₄ haloalkyl, C₁-C₃ alkoxy and hydroxyl;
R₆ is hydrogen or selected from the group consisting of C₁-C₆ alkyl and benzyl;
R₇ and R₈ are independently hydrogen or selected from the group consisting of C₁-C₆ alkyl, C₂-C₆ hydroxyalkyl, (C₂-C₆ alkyl)-N(R₉)(R₁₀), and C₃-C₆ cycloalkyl, or R₇ and R₈ together with the nitrogen to which they are attached represent a nitrogen containing 4- to 7-membered heterocycloalkyl, wherein said 4- to 7-membered nitrogen containing heterocycloalkyl is optionally substituted with C₁-C₃ alkyl, —S(═O)₂(C₁-C₃ alkyl) and —C(═O)O(C₁-C₄ alkyl);
R₉ and R₁₀ are independently hydrogen or C₁-C₃ alkyl, or R₉ and R₁₀ together with the nitrogen to which they are attached represent a nitrogen containing 4- to 7-membered heterocycloalkyl group;
R₁₁ is hydrogen, cyano or C(=O)(C₁-C₃ haloalkyl).

### Regarding the Definition of R₁

In some embodiments of the first and second aspects, provided is the compound of Formula (I) or Formula (II) described above, wherein R₁ is a phenyl which is optionally substituted with one, two or three substituents, wherein each substituent is independently selected from the group consisting of F, Cl, C₁-C₆ alkyl, C₃-C₆ cycloalkyl, C₁-C₃ haloalkyl, C₂-C₄ alkenyl, C₂-C₄ alkynyl, aryl, -(C₁-C₆ alkyl)-aryl, -aryl-(C₁-C₆ alkyl), hydroxyl, cyano, C₁-C₄ hydroxyalkyl, C₁-C₄ alkoxy, -O(C₂-C₄ alkenyl), C₁-C₄ haloalkoxy, C₃-C₆ cycloalkoxy, aryl, —O—aryl, cyano, —O—C(═O)—(C₁-C₆ alkyl), —SH, —S—(C₁-C₆ alkyl), —S—(C₂-C₆ alkenyl), —S(═O)₂(C₁-C₆ alkyl), —S(═O)₂—(C₂-C₆ alkenyl), —N(O)₂, —P(═O)(C₁-C₃ alkyl)₂.

In some preferable embodiments of the first and second aspects, provided is the compound of Formula (I) or Formula (II) described above, wherein R₁ is a phenyl which is optionally substituted with one, two or three substituents, wherein each substituent is independently selected from the group consisting of F, Cl and C₁-C₆ alkyl.

In some more preferable embodiment of the first and second aspects, provided is the compound of Formula (I) or Formula (II) described above, wherein R₁ is

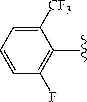

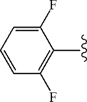

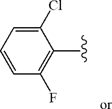

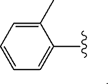

In some embodiments of the first and second aspects, provided is the compound of Formula (I) or Formula (II) described above, wherein R₁ is C₅-C₈ alkyl, C₂-C₆ haloalkyl, or C₄-C₇ cycloalkyl which is optionally partially unsaturated and which is optionally substituted with one or two substituents, wherein each substituent independently selected from F, Cl, phenyl and hydroxyl, and said phenyl substituent is optionally substituted with one, two or three substituents which selected from F, Cl, C₁-C₃ alkyl, C₁-C₄ haloalkyl, C₁-C₃ alkoxy and hydroxy.

In some embodiments of the first and second aspects, provided is the compound of Formula (I) or Formula (II) described above, wherein R₁ is C₁-C₆ alkyl which is substituted with C₃-C₆ cycloalkyl, C₂-C₆ alkyl which is substituted with cyano, hydroxyl, phenyl or C₃-C₈ heterocycloalkyl, 4-7-membered heterocycloalkyl which is optionally substituted with one or two substituents, wherein each substituent is independently selected from C₁-C₃ alkyl, 5-to 6-membered heteroaryl, —C(═O)O(C₁-C₄-alkyl), —C(═O)(C₁-C₃-alkyl), and —C(═O)(C₃-C₆-cycloalkyl).

### Regarding the Definition of R₂

In some embodiments of the first and second aspects, provided is the compound of Formula (I) or Formula (II) described above, wherein R₂ is H or F or CN.

In some preferable embodiments of the first and second aspects, provided is the compound of Formula (I) or Formula (II) described above, wherein R₂ is F.

### Regarding the Definition of R₃

In some embodiments of the first and second aspects, provided is the compound of Formula (I) or Formula (II) described above, wherein R₃ is selected from the group consisting of C₁-C₃ alkyl, C₃-C₆ cycloalkyl, C₁-C₃ hydroxyalkyl, (C₁-C₃ alkoxy)-(C₁-C₆ alkyl), C₁-C₆ alkoxy, —C(═O)OH, and —S(═O)(═NH)(C₁-C₃ alky).

In some preferable embodiments of the first and second aspects, provided is the compound of Formula (I) or Formula (II) described above, wherein R₃ is C₁-C₆ hydroxyalkyl or —CONH₂.

In some more preferable embodiments of the first and second aspects, provided is the compound of Formula (I) or Formula (II) described above, wherein R₃ is —CH₂OH, —CH(OH)CH₃, or —CONH₂.

### Regarding the Definition of R₄

In some embodiments of the first and second aspects, provided is the compound of Formula (I) or Formula (II) described above, wherein R₄ is selected from the group consisting of C₁-C₆ alkyl, C₂-C₆ alkylenyl, C₃-C₆ cycloalkyl, C₂-C₆ haloalkyl and C₂-C₆ hydroxyalkyl.

In some preferable embodiments, provided is the compound of Formula (I) or Formula (II) described above, wherein R₄ is selected from the group consisting of C₁-C₆ alkyl and C₂-C₆ haloalkyl.

In some more preferable embodiments, provided is the compound of Formula (I) or Formula (II) described above, wherein R₄ is selected from the group consisting of CH₃CH₂-and CF₃CH₂—.

### Regarding the Definition of R₅

In some embodiments, provided is the compound of Formula (I) or Formula (II) described above, wherein R₅ is selected from the group consisting of C₁-C₆ alkyl, C₃-C₈ cycloalkyl, C₁-C₆ haloalkyl, C₁-C₆ hydroxyalkyl, C₂-C₆ alkenyl, C₂-C₆ alkynyl, C₄-C₈ cycloalkenyl and phenyl; wherein said phenyl is substituted with one, two, three or four substituents, each substituent is selected from the group consisting of F, Cl, C₁-C₃ alkyl, C₁-C₄ haloalkyl, C₁-C₃ alkoxy and hydroxyl.

In some preferable embodiments of the first and second aspects, provided is the compound of Formula (I) or Formula (II) described above, wherein R₅ is C₁-C₆ alkyl, C₃-C₈ cycloalkyl-C₁-C₆ alkyl or C₁-C₆ haloalkyl.

In some embodiments, provided is the compound of Formula (I) or Formula (II) described above, wherein if feasible, the carbon atom in R₅ which is attached to the core is a secondary carbon atom.

In some preferable embodiments of the first and second aspects, provided is the compound of Formula (I) or Formula (II) described above, wherein R₅ is C₃-C₆ alkyl, C₃-C₈ cycloalkyl-C₂-C₆ alkyl or C₃-C₆ haloalkyl, and the carbon atom in R₅ which is attached to the core is a secondary carbon atom.

In some more preferable embodiments of the first and second aspects, provided is the compound of Formula (I) or Formula (II) described above, wherein R₅ is (CH₃)₂CH—, CH₃CH(CF₃)—, CH₃CH(cyclopropyl)- or CH₃(CH₂)₃CH(CH₃)—.

In some embodiments of the first and second aspects, provided compounds as listed in the Table 1.

TABLE 1

Representative Compounds of Formula (I) or Formula (II)

| Compound | Structure | Name |
|---|---|---|
| 1 | 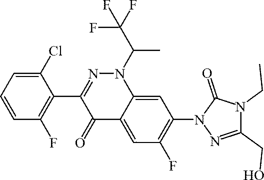 [SMILES: CCn1c(CO)nn(-c2cc3c(cc2F)c(=O)c(-c2c(F)cccc2Cl)nn3C(C)C(F)(F)F)c1=O] | 3-(2-chloro-6-fluorophenyl)-7-(4-ethyl-3-(hydroxymethyl)-5-oxo-4,5-dihydro-1H-1,2,4-triazol-1-yl)-6-fluoro-1-(1,1,1-trifluoropropan-2-yl)cinnolin-4(1H)-one |
| 2 | 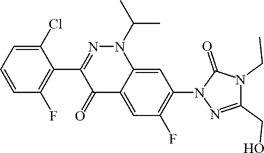 [SMILES: CCn1c(CO)nn(-c2cc3c(cc2F)c(=O)c(-c2c(F)cccc2Cl)nn3C(C)C)c1=O] | 3-(2-chloro-6-fluorophenyl)-7-(4-ethyl-3-(hydroxymethyl)-5-oxo-4,5-dihydro-1H-1,2,4-triazol-1-yl)-6-fluoro-1-isopropylcinnolin-4(1H)-one |
| 3 | 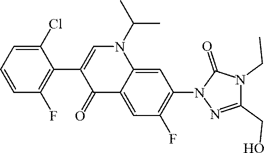 [SMILES: CCn1c(CO)nn(-c2cc3c(cc2F)c(=O)c(-c2c(F)cccc2Cl)cn3C(C)C)c1=O] | 3-(2-chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one |
| 4 | 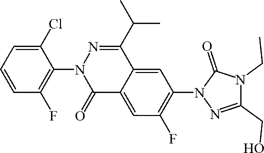 [SMILES: CCn1c(CO)nn(-c2cc3c(C(C)C)nn(-c4c(F)cccc4Cl)c(=O)c3cc2F)c1=O] | 2-(2-chloro-6-fluorophenyl)-6-(4-ethyl-3-(hydroxymethyl)-5-oxo-4,5-dihydro-1H-1,2,4-triazol-1-yl)-7-fluoro-4-isopropylphthalazin-1(2H)-one |
| 5 | 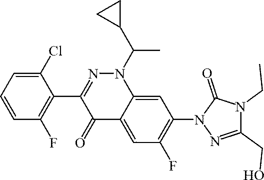 [SMILES: CCn1c(CO)nn(-c2cc3c(cc2F)c(=O)c(-c2c(F)cccc2Cl)nn3C(C)C2CC2)c1=O] | 3-(2-chloro-6-fluorophenyl)-1-(1-cyclopropylethyl)-7-(4-ethyl-3-(hydroxymethyl)-5-oxo-4,5-dihydro-1H-1,2,4-triazol-1-yl)-6-fluorocinnolin-4(1H)-one |
| 6 | 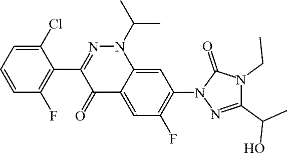 [SMILES: CCn1c(C(C)O)nn(-c2cc3c(cc2F)c(=O)c(-c2c(F)cccc2Cl)nn3C(C)C)c1=O] | 3-(2-chloro-6-fluorophenyl)-7-(4-ethyl-3-(1-hydroxyethyl)-5-oxo-4,5-dihydro-1H-1,2,4-triazol-1-yl)-6-fluoro-1-isopropylcinnolin-4(1H)-one |
| 7 | 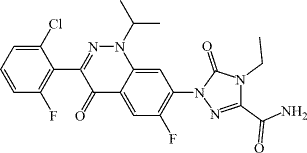 [SMILES: CCn1c(C(N)=O)nn(-c2cc3c(cc2F)c(=O)c(-c2c(F)cccc2Cl)nn3C(C)C)c1=O] | 1-(3-(2-chloro-6-fluorophenyl)-6-fluoro-1-isopropyl-4-oxo-1,4-dihydrocinnolin-7-yl)-4-ethyl-5-oxo-4,5-dihydro-1H-1,2,4-triazole-3-carboxamide |
| 8 | 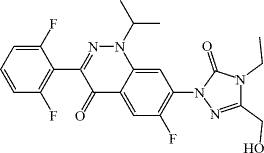 [SMILES: CCn1c(CO)nn(-c2cc3c(cc2F)c(=O)c(-c2c(F)cccc2F)nn3C(C)C)c1=O] | 3-(2,6-difluorophenyl)-7-(4-ethyl-3-(hydroxymethyl)-5-oxo-4,5-dihydro-1H-1,2,4-triazol-1-yl)-6-fluoro-1-isopropylcinnolin-4(1H)-one |
| 9 | 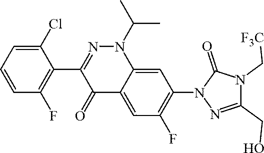 [SMILES: CC(C)n1nc(-c2c(F)cccc2Cl)c(=O)c2cc(F)c(-n3nc(CO)n(CC(F)(F)F)c3=O)cc21] | 3-(2-chloro-6-fluorophenyl)-6-fluoro-7-(3-(hydroxymethyl)-5-oxo-4-(2,2,2-trifluoroethyl)-4,5-dihydro-1H-1,2,4-triazol-1-yl)-1-isopropylcinnolin-4(1H)-one |
| 10 | 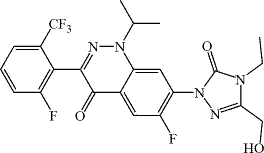 [SMILES: CCn1c(CO)nn(-c2cc3c(cc2F)c(=O)c(-c2c(F)cccc2C(F)(F)F)nn3C(C)C)c1=O] | 7-(4-ethyl-3-(hydroxymethyl)-5-oxo-4,5-dihydro-1H-1,2,4-triazol-1-yl)-6-fluoro-3-(2-fluoro-6-(trifluoromethyl)phenyl)-1-isopropylcinnolin-4(1H)-one |
| 11 | 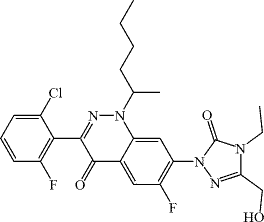 [SMILES: CCCCC(C)n1nc(-c2c(F)cccc2Cl)c(=O)c2cc(F)c(-n3nc(CO)n(CC)c3=O)cc21] | 3-(2-chloro-6-fluorophenyl)-7-(4-ethyl-3-(hydroxymethyl)-5-oxo-4,5-dihydro-1H-1,2,4-triazol-1-yl)-6-fluoro-1-(hexan-2-yl)cinnolin-4(1H)-one |
| 12 | 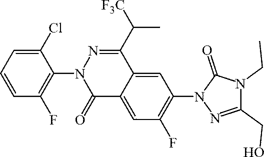 [SMILES: CCn1c(CO)nn(-c2cc3c(C(C)C(F)(F)F)nn(-c4c(F)cccc4Cl)c(=O)c3cc2F)c1=O] | 2-(2-chloro-6-fluorophenyl)-6-(4-ethyl-3-(hydroxymethyl)-5-oxo-4,5-dihydro-1H-1,2,4-triazol-1-yl)-7-fluoro-4-(1,1,1-trifluoropropan-2-yl)phthalazin-1 (2H)-one |
| 13 | 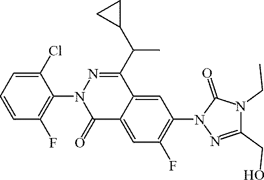 [SMILES: CCn1c(CO)nn(-c2cc3c(C(C)C4CC4)nn(-c4c(F)cccc4Cl)c(=O)c3cc2F)c1=O] | 2-(2-chloro-6-fluorophenyl)-4-(1-cyclopropylethyl)-6-(4-ethyl-3-(hydroxymethyl)-5-oxo-4,5-dihydro-1H-1,2,4-triazol-1-yl)-7-fluorophthalazin-1 (2H)-one |
| 14 | 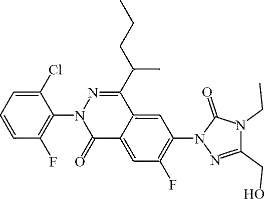 [SMILES: CCCC(C)c1nn(-c2c(F)cccc2Cl)c(=O)c2cc(F)c(-n3nc(CO)n(CC)c3=O)cc12] | 2-(2-chloro-6-fluorophenyl)-6-(4-ethyl-3-(hydroxymethyl)-5-oxo-4,5-dihydro-1H-1,2,4-triazol-1-yl)-7-fluoro-4-(pentan-2-yl)phthalazin-1(2H)-one |
| 15 | 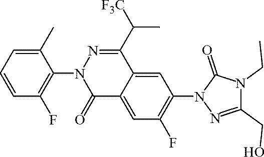 [SMILES: CCn1c(CO)nn(-c2cc3c(C(C)C(F)(F)F)nn(-c4c(C)cccc4F)c(=O)c3cc2F)c1=O] | 6-(4-ethyl-3-(hydroxymethyl)-5-oxo-4,5-dihydro-1H-1,2,4-triazol-1-yl)-7-fluoro-2-(2-fluoro-6-methylphenyl)-4-(1,1,1-trifluoropropan-2-yl)phthalazin-1(2H)-one |
| 16 | 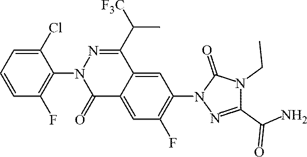 [SMILES: CCn1c(C(N)=O)nn(-c2cc3c(C(C)C(F)(F)F)nn(-c4c(F)cccc4Cl)c(=O)c3cc2F)c1=O] | 1-(2-(2-chloro-6-fluorophenyl)-7-fluoro-1-oxo-4-(1,1,1-trifluoropropan-2-yl)-1,2-dihydrophthalazin-6-yl)-4-ethyl-5-oxo-4,5-dihydro-1H-1,2,4-triazole-3-carboxamide |
| 17 | 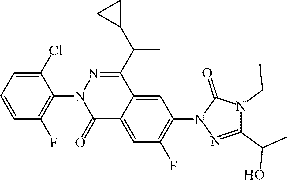 [SMILES: CCn1c(C(C)O)nn(-c2cc3c(C(C)C4CC4)nn(-c4c(F)cccc4Cl)c(=O)c3cc2F)c1=O] | 2-(2-chloro-6-fluorophenyl)-4-(1-cyclopropylethyl)-6-(4-ethyl-3-(1-hydroxyethyl)-5-oxo-4,5-dihydro-1H-1,2,4-triazol-1-yl)-7-fluorophthalazin-1(2H)-one |
| 18 | 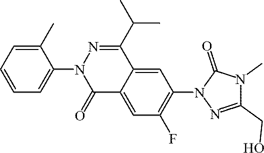 [SMILES: Cc1ccccc1-n1nc(C(C)C)c2cc(-n3nc(CO)n(C)c3=O)c(F)cc2c1=O] | 7-fluoro-6-(3-(hydroxymethyl)-4-methyl-5-oxo-4,5-dihydro-1H-1,2,4-triazol-1-yl)-4-isopropyl-2-(o-tolyl)phthalazin-1(2H)-one |
| 19 | 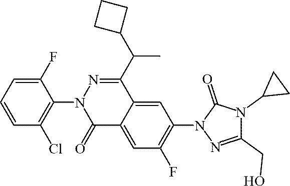 [SMILES: CC(c1nn(-c2c(F)cccc2Cl)c(=O)c2cc(F)c(-n3nc(CO)n(C4CC4)c3=O)cc12)C1CCC1] | 2-(2-chloro-6-fluorophenyl)-4-(1-cyclobutylethyl)-6-(4-cyclopropyl-3-(hydroxymethyl)-5-oxo-4,5-dihydro-1H-1,2,4-triazol-1-yl)-7-fluorophthalazin-1 (2H)-one |
| 20 | 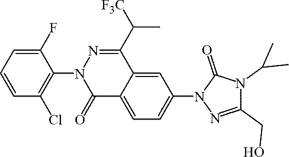 [SMILES: CC(C)n1c(CO)nn(-c2ccc3c(=O)n(-c4c(F)cccc4Cl)nc(C(C)C(F)(F)F)c3c2)c1=O] | 2-(2-chloro-6-fluorophenyl)-6-(3-(hydroxymethyl)-4-isopropyl-5-oxo-4,5-dihydro-1H-1,2,4-triazol-1-yl)-4-(1,1,1-trifluoropropan-2-yl)phthalazin-1(2H)-one |
| 21 | 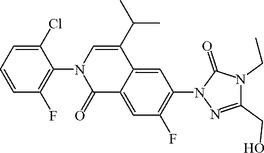 [SMILES: CCn1c(CO)nn(-c2cc3c(C(C)C)cn(-c4c(F)cccc4Cl)c(=O)c3cc2F)c1=O] | 2-(2-chloro-6-fluorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one |
| 22 | 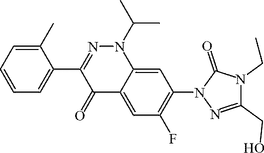 [SMILES: CCn1c(CO)nn(-c2cc3c(cc2F)c(=O)c(-c2ccccc2C)nn3C(C)C)c1=O] | 7-(4-ethyl-3-(hydroxymethyl)-5-oxo-4,5-dihydro-1H-1,2,4-triazol-1-yl)-6-fluoro-1-isopropyl-3-(o-tolyl)cinnolin-4(1H)-one |

In another aspect, provided a pharmaceutical composition comprising: (i) a compound of Formula (I) or Formula (II), or a pharmaceutically acceptable salt, prodrug, or solvate thereof; and (ii) one or more pharmaceutically acceptable carriers. The compound or the pharmaceutical composition can be formulated for any suitable formulations to adapt to any suitable administration manners. For example, the compound or the pharmaceutical composition can be administered via oral, parenteral, pulmonary, nasal, sublingual, lingual, buccal, rectal, vaginal, dermal, transdermal route and so on. Correspondingly, the compound or the pharmaceutical composition can be formulated to different formulations. For example, tablets including disintegrating tablets and sugar-coated tablets, capsules, granules, pellets, powders, emulsions, suspensions, syrups, aerosols or solutions are available formulations.

The compound or the pharmaceutical compositions may be administered in either single or multiple doses. A compound of Formula (I) or Formula (II), or a pharmaceutically acceptable salt, prodrug, or solvate salt thereof can be formulated so as to provide the desired release schedule of the active ingredient based on the therapeutic treatment purpose.

The pharmaceutical composition is preferably made in the form of a dosage unit containing a particular amount of the active ingredient in the form of tablets, pills, powders, suspensions, emulsions, solutions, syrups, and capsules. For example, these may contain an amount of active ingredient from about 0.1 to 1000 mg, preferably from about 0.1 to 500 mg. A suitable daily dose for a human or other mammal may vary widely depending on the condition of the patient and other factors, but, once again, can be determined using routine methods. The daily dose can be administered in one to four doses per day. For therapeutic purposes, the active compounds of this invention are ordinarily combined with one or more adjuvants appropriate to the indicated route of administration drops suitable for administration to the eye, ear, or nose. A suitable topical dose of active ingredient of a compound of the invention is 0.1 mg to 150 mg administered one to four, preferably one or two times daily. For topical administration, the active ingredient may comprise from 0.001 % to 10% w/w, e.g. from 1 % to 2% by weight of the formulation, preferably not more than 5% w/w, and more preferably from 0.1 % to 1 % of the formulation.

In a particular embodiment, an initial daily dose of about 0.1 to 500 mg of a compound of Formula (I) or Formula (II) is administered to the subject and increasing the dose by increments until clinical efficacy is achieved. Increments of about 5, 10, 25, 50, or 100 mg can be used to increase the dose. The dosage can be increased daily, every other day, twice per week, or once per week.

### Methods and Uses

Compounds disclosed can be utilized to inhibit the activity of DHODH. In one aspect, provided is a method of inhibiting activity of DHODH, comprising administering a subject in need an effective amount of the compound of Formula (I) or Formula (II), or a pharmaceutically acceptable salt, prodrug, solvate thereof. Compounds disclosed can effectively inhibit DHODH and therefore can be used for the treatment or prophylaxis of diseases, preferably viral infection, cancer or inflammatory disorders in human and animals.

In one aspect, provided a use of the compound of Formula (I) or Formula (II), or a pharmaceutically acceptable salt, prodrug, solvate thereof for the treatment or prophylaxis of virus infection. Examples of virus can be DNA virus or RNA virus, including but not limited to influenza A virus, influenza B virus, Zika virus, Ebola virus, Rhinovirus, Enterovirus 71, and the novel existed cornavirus SARS-CoV-2. A subject having or at risk of developing a virus infection can be administered an effective amount of the compound disclosed.

In another aspect, provided a use of the compound of Formula (I) or Formula (II), or a pharmaceutically acceptable salt, prodrug, solvate thereof for the treatment of cancer. Examples of cancer include, but not limited to colorectal carcinoma, leukemia, lymphoma, breast cancer, small-cell and non-small-cell lung carcinoma, liver cancer, lung cancer, ovarian cancer, pancreatic cancer, renal cell carcinoma, gastric cancer, skin cancer. Cancer associated with DHODH can be treated by the compounds disclosed. Leukemias include but not limited to acute lymphoblastic leukemia(ALL), acute myeloid leukemia(AML), acute T-cell leukemia, acute monocytic leukemia, acute promyelocytic leukemia(APL), chronic myelogenous leukemia and chronic myeloid leukemia. Lymphomas include, but are not limited to AIDS-related lymphoma, chronic lymphocytic lymphoma (CLL), non-Hodgkin’s lymphoma (NHL), T-non-Hodgkin lymphoma (T-NHL), activated B-cell DLBCL, germinal center B-cell lymphoma DLBCL, double-hit lymphoma and double-expressor lymphoma; anaplastic large cell lymphoma, B-cell lymphoma, cutaneous T-cell lymphoma, Burkitt’s lymphoma, follicular lymphoma, hairy cell lymphoma, Hodgkin’s disease, mantle cell lymphoma (MCL) and chronic lymphocytic lymphoma.

The compounds disclosed can be used in combination with other anti-cancer agents. In addition, the compounds disclosed can be used with different cure methods, such as chemotherapy, radiotherapy or surgical intervention.

In another aspect, provided is a use of the compound of Formula (I) or Formula (II), or a pharmaceutically acceptable salt, prodrug, solvate thereof for the treatment of inflammatory disorders. Example of inflammatory disorders include but not limited to systemic anaphylaxis and hypersensitivity responses, drug allergies, insect sting allergies, inflammatory bowel diseases such as ulcerative colitis and ileitis, asthma and respiratory allergic diseases such as allergic asthma, allergic rhinitis, allergic conjunctivitis and hypersensitivity lung diseases.

In another aspect, provided is a method of inhibiting cell proliferation comprising contacting the cell with a compound of Formula (I) or Formula (II), or a pharmaceutically acceptable salt, prodrug, solvate thereof. The cell can be in vitro or in vivo.

In another aspect, provided is a method of preparing the compound of Formula (I) or Formula (II). In some specific embodiments, provided is a method of preparing the compound 2, comprising:
(a) 3-bromo-4-fluoro-aniline reacts with 2-(2-chloro-6-fluoro-phenyl)acetonitrile to produce 1-(2-amino-4-bromo-5-fluoro-phenyl)-2-(2-chloro-6-fluoro-phenyl)ethanone;
(b) 1-(2-amino-4-bromo-5-fluoro-phenyl)-2-(2-chloro-6-fluoro-phenyl)ethanone under NaNO₂ and acid to produce 7-bromo-3-(2-chloro-6-fluoro-phenyl)-6-fluoro-1H-cinnolin-4-one;
(c) 7-bromo-3-(2-chloro-6-fluoro-phenyl)-6-fluoro-1H-cinnolin-4-one reacts with 2-iodopropane to produce 7-bromo-3-(2-chloro-6-fluoro-phenyl)-6-fluoro-1-isopropylcinnolin-4(1H)-one;
(d) 7-bromo-3-(2-chloro-6-fluoro-phenyl)-6-fluoro-1-isopropylcinnolin-4(1H)-one reacts with 4-ethyl-3-(hydroxymethyl)-1H-1,2,4-triazol-5-one to produce compound 2.

### EXAMPLES

### Synthesis of Compounds

### Method 1: Preparation of 3-(2-chloro-6-fluorophenyl)-7-(4-ethyl-3-(hydroxymethyl)-5-oxo-4,5-dihydro-1H-1,2,4-triazol-1-yl)-6-fluoro-1-(1,1,1-trifluoropropan-2-yl)cinnolin-4(1H)-one (1)

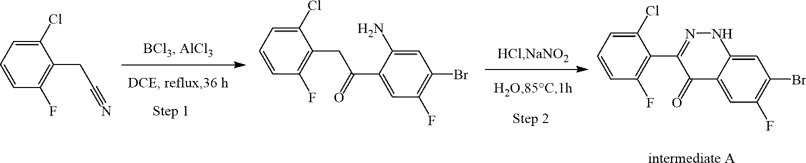

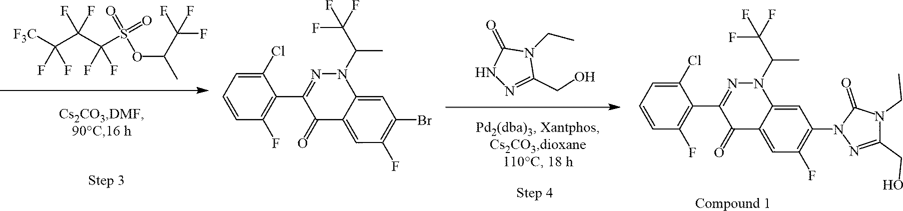

Step 1. To a solution of BCl₃ (34.2 mL, 36.0 mmol, 1 M in DCM) in DCE (60.0 mL) was added 3-bromo-4-fluoroaniline (7.00 g, 33.8 mmol) at 0° C. and it was stirred at 0° C. for 1 hour. To the mixture was added 2-(2-chloro-6-fluorophenyl)acetonitrile (10.0 g, 84.0 mmol) and AlCl₃ (5.00 g, 36.0 mmol) at 0° C. The mixture was stirred at 90° C. for 36 hours. The mixture was cooled to room temperature. The mixture was quenched with aq. HCI solution (2 N, 32.0 mL) and then stirred at 80° C. for another 1 hour. The mixture was poured into ice water (200 mL) and extracted with DCM (100 mL × 3). The combined organic layer was dried over sodium sulfate anhydrous, filtered and concentrated in vacuum to give the crude product. The residue was purified by column chromatography (silica gel, DCM: MeOH = 30:1) to give 1-(2-amino-4-bromo-5-fluorophenyl)-2-(2-chloro-6-fluorophenyl)ethan-1-one (2.20 g, 18.1% yield) as a yellow solid. LC-MS (ESI) [M+H]⁺ 361.9.

Step 2. To a solution of 1-(2-amino-4-bromo-5-fluorophenyl)-2-(2-chloro-6-fluorophenyl)ethan-1-one (2.20 g, 6.12 mmol) in aq. HCI solution (5 N, 25.0 mL) was slowly added a solution of NaNO: (700 mg, 10.1 mmol) in H₂O (5.00 mL) at 0° C. and it was stirred at 85° C. for 1.5 hours. The mixture was cooled to room temperature. The mixture was adjusted to pH 8 with sat. NaHCO₃ solution and extracted with DCM (50 mL × 3). The combined organic layer was dried over sodium sulfate anhydrous and concentrated in vacuum to give the crude product. The residue was purified by column chromatography (silica gel, DCM: MeOH = 20:1) to give 7-bromo-3-(2-chloro-6-fluorophenyl)-6-fluorocinnolin-4(1H)-one (intermediate A, 800 mg, 35.4% yield) as a yellow solid. LC-MS (ESI) [M+H]⁺ 372.9.

### Method 1 Includes Step 3 and Step 4:

Step 3. To a suspension of 7-bromo-3-(2-chloro-6-fluorophenyl)-6-fluorocinnolin-4(1H)-one (300 mg, 0.806 mmol) and Cs₂CO₃ (786 mg, 2.42 mmol) in DMF (3.00 mL) was added 1,1,1-trifluoropropan-2-yl 1,1,2,2,3,3,4,4,4-nonafluorobutane-1-sulfonate (2.27 g crude, 4.03 mmol, purity: ~70.0%) and it was stirred at 90° C. for 16 hours. The mixture was cooled to room temperature. The mixture was diluted with H₂O (15.0 mL) and extracted with ethyl acetate (20 mL × 3). The combined organic layer was dried over sodium sulfate anhydrous and concentrated in vacuum to give the residue, which was purified by prep-TLC (silica gel, DCM: MeOH = 20:1) to give 7-bromo-3-(2-chloro-6-fluorophenyl)-6-fluoro-1-(1,1,1-trifluoropropan-2-yl)cinnolin-4(1H)-one (60 mg, 15.9% yield) as a yellow solid. LC-MS (ESI) [M+H]⁺ 468.7.

Step 4. To a solution of 7-bromo-3-(2-chloro-6-fluorophenyl)-6-fluoro-1-(1,1,1-trifluoropropan-2-yl)cinnolin-4(1H)-one (60 mg, 0.13 mmol) in dioxane (2.00 mL) was added 4-ethyl-5-(hydroxymethyl)-2,4-dihydro-3H-1,2,4-triazol-3-one (28 mg, 1.5 eq), Xantphos (9.5 mg, 0.3 eq), Pd₂(dba)₃ (12 mg, 0.1 eq) and Cs₂CO₃ (88 mg, 2.1 eq) at room temperature. The mixture was stirred at 110° C. for 18 hours. The mixture was cooled to room temperature. The mixture was diluted with water (20.0 mL), extracted with ethyl acetate (20.0 mL * 3). The combined organic layer was washed with brine, dried over sodium sulfate anhydrous, filtered and concentrated under reduced pressure. The residue was purified by prep-HPLC to give compound 1 (12.0 mg, 17.7% yield) as a yellow solid. LC-MS (ESI) [M+H]⁺ 529.8. ¹H NMR (400 MHz, DMSO-d₆): δ 8.53 (d, J = 6.0 Hz, 1H), 8.10 (dd, J = 10.0 Hz, 2.4 Hz, 1H), 7.60 -7.57 (m, 1H), 7.51 - 7.49 (m, 1H), 7.42 - 7.37 (m, 1H), 6.30 - 6.20 (m, 1H), 5.85 (t, J = 6.0 Hz, 1H), 4.53 (d, J = 5.6 Hz, 2H), 3.81 (q, J = 7.2 Hz, 2H), 1.73 - 1.69 (m, 3H), 1.31 (t, J = 7.2 Hz, 3H).

### Method 2: Preparation of 3-(2-chloro-6-fluorophenyl)-7-(4-ethyl-3-(hydroxymethyl)-5-oxo-4,5-dihydro-1H-1,2,4-triazol-1-yl)-6-fluoro-1-isopropylcinnolin-4(1H)-one (2)

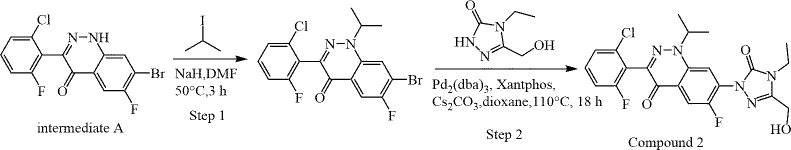

Step 1. To a suspension of 7-bromo-3-(2-chloro-6-fluorophenyl)-6-fluorocinnolin-4(1H)-one (intermediate A, 150 mg, 0.405 mmol) was added NaH (33.0 mg, 0.810 mmol, 60%) at 0° C. and stirred at 50° C. for 1 h. To the mixture was added 2-iodopropane (275 mg, 1.62 mmol) and stirred at 50° C. for 3 hours. The mixture was cooled to room temperature. The mixture was quenched with H₂O (15.0 mL) at 0° C. and extracted with ethyl acetate (20 mL × 3). The combined organic layer was dried over sodium sulfate anhydrous and concentrated in vacuum to give the residue, which was purified by prep-TLC (silica gel, DCM: MeOH = 20:1) to give 7-bromo-3-(2-chloro-6-fluorophenyl)-6-fluoro-1-isopropylcinnolin-4(1H)-one (60 mg, 35.9% yield) and 7-bromo-3-(2-chloro-6-fluorophenyl)-6-fluoro-4-isopropoxycinnoline (80 mg, 47.9% yield) as a yellow solid. LC-MS (ESI) [M+H]⁺ 414.9.

Step 2. In a similar manner as the preparation of compound 1, 3-(2-chloro-6-fluorophenyl)-7-(4-ethyl-3-(hydroxymethyl)-5-oxo-4,5-dihydro-1H-1,2,4-triazol-1-yl)-6-fluoro-1-isopropylcinnolin-4(1H)-one (compound 2) was synthesized (11.0 mg, 15.9% yield) as a yellow solid. LC-MS (ESI) [M+H]⁺ 475.9. ¹H NMR (400 MHz, DMSO-d₆): δ 8.37 (d, J = 6.4 Hz, 1H), 8.06 (d, J = 10.0 Hz, 1H), 7.58 - 7.48 (m, 1H), 7.50 - 7.48 (m, 1H), 7.40 - 7.35 (m, 1H), 5.87 - 5.81 (m, 2H), 5.29 - 5.26 (m, 1H), 4.53 (d, J = 4.0 Hz, 2H), 3.83 (q, J = 7.2 Hz, 2H), 1.47 - 1.44 (m, 6H), 1.31 (t, J = 7.2 Hz, 3H).

### Method 3: Preparation of 3-(2-chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one (3)

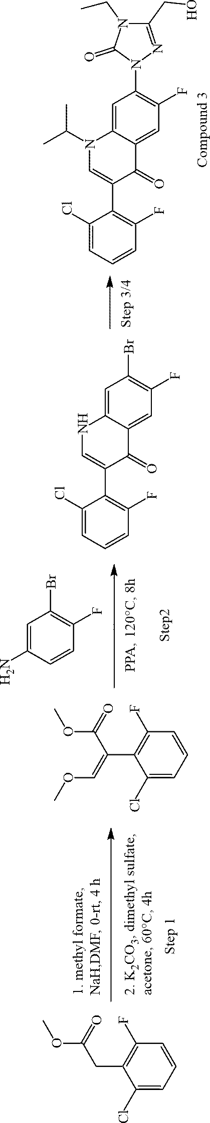

Step 1. To a solution of methyl 2-(2-chloro-6-fluorophenyl)acetate (5.00 g, 24.7 mmol) in DMF (50.0 mL) was added NaH (1.18 g, 29.5 mmol) at 0° C. 30 min later, the solution was added with 5.00 mL DMF solution of methyl formate (5.00 mL). The mixture was stirred at room temperature for 4 hours. The mixture was diluted with ammonium chloride aqueous solution (200 mL), extracted with ethyl acetate (100 mL * 3). The combined organic layer was washed with brine, dried over sodium sulphate anhydrous, filtered and concentrated under reduced pressure. The residue was dissolved in acetone (40.0 mL) and the solution was added K₂CO₃ (10.2 g, 73.9 mmol) and dimethyl sulfate (2.00 mL) at room temperature. The mixture was heated to reflux for 4 hours. The mixture was cooled to room temperature and filtered, the filtrate was concentrated under reduced pressure. The residue was purified by column chromatography (PE/EA=10/1) to give methyl (Z)-2-(2-chloro-6-fluorophenyl)-3-methoxyacrylate (3.00 g, 50.0% yield) as colorless oil. ¹H NMR (400 MHz, CDCl₃) δ 7.63 (d, J = 4.8 Hz, 1H), 7.26 - 7.21 (m, 2H), 7.02 - 6.98 (m, 1H), 3.87 (s, 3H), 3.72 (s, 3H).

Step 2. A solution of methyl (Z)-2-(2-chloro-6-fluorophenyl)-3-methoxyacrylate (3.00 g, 12.3 mmol) and 3-bromo-4-fluoroaniline (2.56 g, 13.5 mmol) in PPA (20.0 mL) was added heated to 120° C. for 8 hours. The mixture was cooled to 80° C. and diluted with water (100 mL). The mixture was adjusted pH to 8 with sodium carbonate aqueous solution, extracted with ethyl acetate (20.0 mL * 3). The combined organic layer was washed with brine, dried over sodium sulphate anhydrous, filtered and concentrated under reduced pressure. The residue was purified by column chromatography (PE/EA=5/1) to give 7-bromo-3-(2-chloro-6-fluorophenyl)-6-fluoroquinolin-4(1H)-one (350 mg, 15.2% yield) as a white solid. ¹H NMR (400 MHz, DMSO-d₆) δ 12.29 (d, J = 5.6 Hz, 1H), 8.19 (d, J = 6.0 Hz, 1H), 7.98 (d, J = 5.6 Hz, 1H), 7.91 (d, J = 8.8 Hz, 1H), 7.50 - 7.37 (m, 2H), 7.30 (t, J = 8.0 Hz, 1H).

Step 3 and 4: To a solution of 7-bromo-3-(2-chloro-6-fluorophenyl)-6-fluoroquinolin-4(1H)-one (50.0 mg, 0.135 mmol) in DMF (1.00 mL) was added NaH (11.0 mg, 0.275 mmol) at room temperature. The mixture was stirred at 50° C. for 1 hour. The solution was added with 0.500 mL DMF solution of 2-iodopropane (115 mg, 0.676 mmol). The mixture was stirred at 50° C. for 5 hours. The mixture was cooled to room temperature. The mixture was diluted with water (20.0 mL), extracted with ethyl acetate (20.0 mL * 3). The combined organic layer was washed with brine, dried over sodium sulphate anhydrous, filtered and concentrated under reduced pressure. The residue was purified by silica gel column chromatography (PE/EA=10/1) to give 7-bromo-3-(2-chloro-6-fluorophenyl)-6-fluoro-1-isopropylquinolin-4(1H)-one (20 mg, 58.8%, peak 2) and 7-bromo-3-(2-chloro-6-fluorophenyl)-6-fluoro-4-isopropoxyquinoline (10 mg, 29.4%, peak 1) as a white solid. LC-MS (ESI) [M+H+2]⁺ 414.0. According to the method described in similar procedure as compound 2, 3-(2-chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one (compound 3) was synthesized from 7-bromo-3-(2-chloro-6-fluorophenyl)-6-fluoro-1-isopropylquinolin-4(1H)-one (3.00 mg, 13.0% yield) as a white solid. LC-MS (ESI) [M+H]⁺ 475.1. ¹H NMR (400 MHz, DMSO-d₆): δ 8.35 (s, 1H), 8.23 (d, J = 6.0 Hz, 1H), 8.07 (d, J = 10.8 Hz, 1H), 7.48 - 7.45 (m, 2H), 7.33 - 7.30 (m, 1H), 5.83 (t, J = 5.6 Hz, 1H), 5.10 -5.02 (m, 1H), 4.52 (d, J = 6.0 Hz, 2H), 3.86 - 3.81 (m, 2H), 1.50 (t, J = 7.2 Hz, 6H), 1.31 (t, J = 7.2 Hz, 3H).

### Method 4: 2-(2-chloro-6-fluorophenyl)-6-(4-ethyl-3-(hydroxymethyl)-5-oxo-4,5-dihydro-1H-1,2,4-triazol-1-yl)-7-fluoro-4-isopropylphthalazin-1(2H)-one (4)

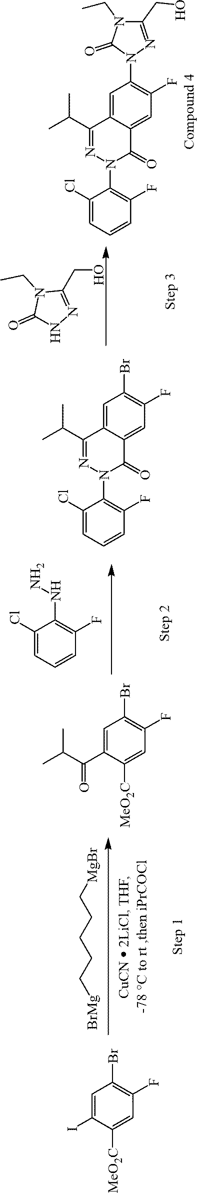

Step 1. LiCl (1.25 g, 29.53 mmol, 604.83 uL, 2 eq) and stir bar were dried under vacuum at 130° C. for 2 hr and then cooled to 22° C. before addition of CuCN (1.32 g, 14.77 mmol, 3.23 mL, 1 eq) followed by THF (14.7 mL). The mixture was stirred for 15 mins. At the same time, a flask with bromo-[5-(bromomagnesio)pentyl]magnesium (0.5 M, 29.53 mL, 1 eq) in THF (59 mL) was cooled to -78° C. and a solution of 1 M CuCN•2LiCl (14.8 mL) was added dropwise and stirred at -78° C. for 30 min. Methyl 4-bromo-5-fluoro-2-iodo-benzoate (5.3 g, 14.77 mmol, 1 eq) in THF (14.7 mL) was added at -78° C. then the reaction mixture was warmed to 22° C. by removing the cooling bath and stirred at 22° C. for 1 hr before 2-methylpropanoyl chloride (2.36 g, 22.15 mmol, 2.31 mL, 1.5 eq) was added and stirred for 1 hr. The reaction mixture was then cooled to 0° C. and quenched with sat. NH₄Cl and extracted with EtOAc (3 × 50 mL). The combined organic layer was dried over Na₂SO₄ and concentrated. The residual was re-dissolved in DCM and passed through a celite plug to remove Mg salts. The filtrate was concentrated to provide a solid which was triturated with hexane/Et20 (95%/5%). The resulting solid was filtered and dried (3.2 g, 10.56 mmol, 71% yield) as a yellow solid.. LC-MS (ESI) [M+H]⁺ 304.

Step 2. A mixture of methyl 4-bromo-5-fluoro-2-(2-methylpropanoyl)benzoate (500 mg, 1.7 mmol, 1 eq) and (2-chloro-6-fluoro-phenyl)hydrazine (1.06 g, 4 eq) was dissolved in MeOH (17 mL) followed by the addition of H₂SO₄ (352 uL, 4 eq). The reaction mixture was heated to 80° C. for 18 hr. The reaction mixture was cooled to 0° C. and diluted with water. The aqueous layer was extracted with EtOAc (3 × 5 mL). The combined organic layers were dried over Na₂SO₄ and concentrated and purified through FCC using Biotage Sfar 10 g silica column with Hexanes/EtOAc (100%/0% to 90%/10%). 6-bromo-2-(2-chloro-6-fluorophenyl)-7-fluoro-4-isopropyl-phthalazin-1-one (70 mg, 169 umol, 10% yield) was isolated as a yellow oil. LC-MS (ESI) [M+H]⁺ 414.

Step 3. Compound 4 was prepared according to the last step of Method 1. LC-MS (ESI) [M+H]⁺ 476.

Also provided another method to prepare compound of Formula (II), such as the compound 2 listed in Table1. The specific preparation methods are as follows:

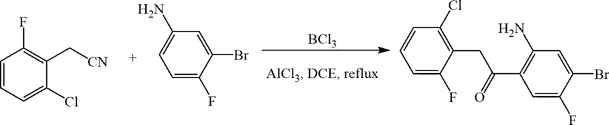

1-amino-4-bromo-5-fluorophenyl)-2-chloro-6-fluorophenyl)ethan-1-one: 3-bromo-4-fluoro-aniline (17.5 g, 92.10 mmol, 1 eq) was dissolved in DCE (184 mL), cooled to 0° C. and BCl₃ (1 M, 98.55 mL, 1.07 eq) was cannulated into the mixture. The mixture was stirred at 0° C. for 1 hr. 2-(2-chloro-6-fluoro-phenyl)acetonitrile (24.99 g, 147.36 mmol, 1.6 eq) and AlCl₃ (13.14 g, 98.55 mmol, 5.39 mL, 1.07 eq) were added at 0° C. After addition, the mixture was heated to reflux for 24 hr. The mixture was cooled to 22° C. and 2 M HCI (100 mL) was added carefully. The mixture was heated to 80° C. for 1 hr. Upon completion, the mixture was cooled to 22° C. and filtered over a pad of celite. The layers from the filtrate were separated. The aqueous layer was extracted with DCM (3 × 100 mL). The combined organic layers were dried over Na₂SO₄ and concentrated. The crude was purified by Biotage Sfar silica column using Hexane/EtOAc gradient yielding 1-(2-amino-4-bromo-5-fluoro-phenyl)-2-(2-chloro-6-fluoro-phenyl)ethanone (7.37 g, 21 mmol, 22% yield) as a yellow solid.

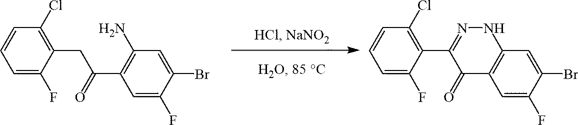

7-bromo(2-chloro-6-fluorophenyl)-6-fluorocinnolin-4(1H)-one: 1-(2-amino-4-bromo-5-fluoro-phenyl)-2-(2-chloro-6-fluoro-phenyl)ethanone (6.94 g, 19.25 mmol, 1 eq) was suspended in 5 N HCI (193 mL) then heated to 40° C. NaNO₂ (1.3 M, 17.77 mL, 1.2 eq) was added over 1 hr to the mixture. After addition, the mixture was stirred at 40° C. for 0.5 hr. The addition of NaNO₂ was repeated 2 more cycles. Upon completion, the mixture was cooled to 22° C. and the resulting precipitate filtered. Solid was triturated with 5 N HCI several times yielding 7-bromo-3-(2-chloro-6-fluoro-phenyl)-6-fluoro-1H-cinnolin-4-one (6.28 g, 16.90 mmol, 87.82% yield) as a yellow solid.

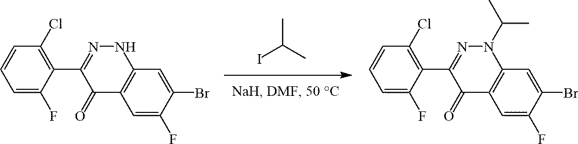

7-bromo(2-chloro-6-fluorophenyl)-6-fluoro-1-isopropylcinnolin-4(1H)-one: 7-bromo(2-chloro-6-fluoro-phenyl)-6-fluoro-1H-cinnolin-4-one (10.4 g, 27.99 mmol, 1 eq) was dissolved in DMF (280 mL) then cooled to 0° C. followed by the addition of NaH (2.24 g, 55.98 mmol, 60% purity, 2 eq). The mixture was heated to 50° C. for 1 hr then 2-iodopropane (19.03 g, 111.96 mmol, 11.20 mL, 4 eq) was added and heated to 50° C. for 1 hr. Upon completion, the mixture was added to brine then EtOAc added. The layer was separated and the aqueous layer was extracted with EtOAc (2 × 100 mL). The combined organic layers were dried over Na₂SO₄ and concentrated via rotatory evaporator under high vacuum. The residual was dissolved in EtOAc and washed with water (3 × 100 mL). The organic layer was dried over Na₂SO₄ and concentrated. The crude was purified by Biotage Sfar 100 g silica column using hexane/EtOAc gradient yielding 7-bromo-3-(2-chloro-6-fluoro-phenyl)-6-fluoro-1-isopropyl-cinnolin-4-one (4 g, 9.67 mmol, 34.55% yield) as a yellow solid.

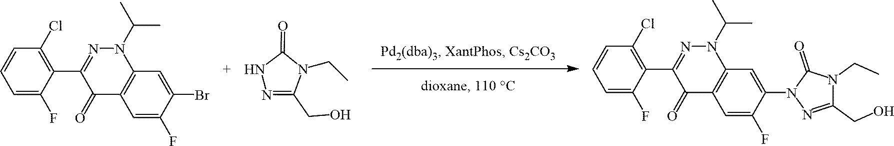

3-chloro-6-fluorophenyl)-7-(4-ethyl-3-(hydroxymethyl)-5-oxo-4,5-dihydro-1H-1,2,4-triazol-1-yl)-6-fluoro-1-isopropylcinnolin-4(1H)-one: 7-bromo-3-chloro-6-fluorophenyl)-6-fluoro-1-isopropyl-cinnolin-4-one (4.73 g, 11.43 mmol, 1 eq), Pd₂(dba)₃ (1.05 g, 1.14 mmol, 0.1 eq), Xantphos (1.98 g, 3.43 mmol, 0.3 eq), 4-ethyl-3-(hydroxymethyl)-1H-1,2,4-triazol-5-one (2.46 g, 17.15 mmol, 1.5 eq), and Cs₂CO₃ (7.45 g, 22.87 mmol, 2 eq) were suspended in dioxane (230 mL). The mixture was purged with nitrogen for 10 min. The mixture was heated to 110° C. for 1 hr. Upon completion, the mixture was cooled to 22° C. The mixture was partitioned between water/EtOAc. Layers were separated and the aqueous layer was extracted with EtOAc (3 × 100 mL). The combined organic layer was dried over Na₂SO₄ and concentrated. The crude was purified by Biotage Sfar silica column using DCM/THF gradient yielding 3-(2-chloro-6-fluoro-phenyl)-7-[4-ethyl-3-(hydroxymethyl)-5-oxo-1,2,4-triazol-1-yl]-6-fluoro-1-isopropyl-cinnolin-4-one (4 g, 8.41 mmol, 73.51% yield) as a light yellow solid. LCMS [M+H]⁺ = 476; ¹H NMR (600 MHz, DMSO-d6) δ ppm 1.32 (t, J=7.15 Hz, 3 H) 1.42 -1.50 (m, 6 H) 3.84 (q, J=7.15 Hz, 2 H) 4.51 - 4.56 (m, 2 H) 5.28 (dt, J=12.79, 6.35 Hz, 1 H) 5.84 (br s, 1 H) 7.38 (t, J=8.76 Hz, 1 H) 7.49 (d, J=8.07 Hz, 1 H) 7.58 (td, J=8.25, 6.24 Hz, 1 H) 8.07 (d, J=10.09 Hz, 1 H) 8.37 (d, J=6.05 Hz, 1 H).

Compounds 5-22 are prepared according to the method 1, 2, 3, or 4.

### Biological Evaluation

### 1. Biochemical Enzymatic Assay

The biochemical activity of DHODH was measured by the bleaching of the dye 2,6-dichlorophenolindophenol (DCIP) (Knecht W. et al., Biochem. Pharmacol. 1998, 56, 1259-1264). The assay was conducted in buffer containing 50 mM Tris, 150 mM KCI, and 0.1% Triton® X-100 (pH 8.0). 5 ng DHODH protein in 50 µL assay buffer was mixed in a well of 96-well clear assay plate (Corning Cat#3599) with 25 µL series concentration of inhibitor at room temperature for 30mins prior to adding 25 µL substrate solution (1 mM L-Dihydroorotic acid, 0.1 mM Decylubiquinone, 0.06 mM DCIP). After incubation at RT for 60mins, absorbance at 600 nm was obtained using a PerkinElmer multimode plate-reading spectrophotometer. Purified recombinant human DHODH was purchased from R&D systems (Catalog# 10062-DD-020), L-dihydroorotic acid (Sigma, D7128), decylubiquinone (Sigma, D7911), DCIP (Sigma, D1878) and other chemicals were purchased from Sigma-Aldrich. Results:

TABLE 2

| Compounds | Biochemical Enzymatic Assay IC₅₀ (nM) |
|---|---|
| Compound 1 | 215 |
| Compound 2 | 97 |
| Compound 4 | 11 |

### 2. MV11 Cells Proliferation Assay

5000 cells/well of MV-4-11 cells were seeded in IMDM 10% fetal calf serum (FBS, BI, Catalog#) in 96-well plates. The next day, cells were incubated with different concentrations of test compounds for 72 h. Cellular viability was analyzed using CellTiter-Glo® Luminescent Cell Viability Assay (Promega, #G7570) according to manufacturer’s instructions.

TABLE 3

| Compounds | MV-4-11 Cell Proliferation Assay IC₅₀ (nM) |
|---|---|
| Compound 2 | 0.09 |
| Compound 4 | 0.9 |

### 3. Anti-viral

The cytopathic effect (CPE) was used to test the antiviral activity of the compound against influenza virus, and the cytotoxicity was also determined. The compound was tested at 8 concentrations, double-replicated. Use CCK-8 reagent to detect cell viability.

MDCK cells were seeded in a microtiter plate and cultured overnight at 37° C. and 5% CO₂. The compound and the virus strain (the A/PR/8/34 (H1N1) strain, the H7N9 strain or the H1N1(XJ49) strain) were added the next day. Set cell (no compound treatment or virus infection) and virus infection (cell infection with virus, no compound treatment) control. The final concentration of DMSO in the cell culture medium is 0.5%. The cells were cultured at 37° C. and 5% CO₂ for 5 days until the cytopathic rate of virus control wells reached 80-95%. The cytotoxicity test is the same as the antiviral test, but there is no virus infection. CCK-8 reagent was used to detect cell viability, and the raw data was used to calculate the compound’s antiviral activity and cytotoxicity. GraphPad Prism software was used to analyze the compound dose-response curve and calculate the EC₅₀ and CC₅₀ values, wherein EC₅₀ means the concentration of the test compound at which half of the virus are effectively inhibited, and CC₅₀ means the concentration of the test compound at which half of the cells are dead.

TABLE 4

Activity of anti-A/PR/8/34 (H1N1)

| Compounds | EC₅₀ (nM) | CC₅₀ (nM) |
|---|---|---|
| compound 4 | 23 | >1000 |
| compound 2 | 64 | >1000 |
| Baloxavir | 1.1 | 18 |
| Oseltamivir acid | 0.15 | >100 |

TABLE 5

Activity of anti-H7N9 and anti-H1N1 (XJ49)

| | Anti-H7N9 EC₅₀ (nM) | anti-H1N1 (XJ49) EC₅₀ (nM) |
|---|---|---|
| compound 4 | 14 | 5.7 |

Vero cells were seeded in a microtiter plate, 50µl of virus (the ZIKV strain) solution was added, and then the compound was added. The final concentration of DMSO in the cell culture medium is 0.5%. The cells were cultured at 37° C. and 5% CO₂ for 3 days. By detecting the expression of the reporter gene, the antiviral activity of the compound is calculated. GraphPad Prism software was used to analyze the compound dose-response curve and calculate the EC50.

TABLE 6

Activity of anti-ZIKV

| | EC₅₀ (nM) |
|---|---|
| Compound 4 | 0.37 |

### 4. THP-1 Cells Proliferation Assay

2000 cells/well of THP-1 cells were seeded in RPMI 1640 with Glutamax (Gibco, #11875-093) and 10% fetal calf serum (FBS, BI, Catalog#) in 384-well plates. The next day, cells were incubated with different concentrations of test compounds for 72 h. Cellular viability was analyzed using CellTiter-Glo® Luminescent Cell Viability Assay (Promega, #G7570) according to manufacturer’s instructions.

The compounds of the present invention exist broad spectrum anti-viral activity.

1. A compound of Formula (I), or a pharmaceutically acceptable salt, stereoisomer, prodrug, or solvate thereof, 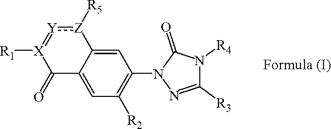
wherein:
X and Z are independently N or C;
Y is selected from the group consisting of N and CR₇;
wherein when X is N, Z is C, 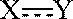
is a single bond, 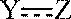
is a double bond;
when X is C, Z is N, 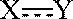
is double bond, 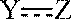
is a single bond;
R₁ is selected from the group consisting of
C₁-C₈ alkyl;
C₂-C₈ haloalkyl;
C₃-C₈ cycloalkyl which is optionally substituted with 1-6 halogen atoms or a group selected from hydroxyl and phenyl, wherein said phenyl substituent is optionally substituted with 1-4 halogen atoms or a group selected from C₁-C₃ alkyl, C₁-C₄ haloalkyl, C₁-C₃ alkoxy, CN and hydroxyl;
C₃-C₆ alkyl which is substituted with a monocyclic- or bicyclic heteroaryl group;
(C₂-C₆ hydroxyalkyl)-O-(C₂-C₆ alky);
(C₃-C₆ alkyl)-N(R₇)(R₈);
(C₃-C₈ cycloalkyl)-N(R₇)(R₈);
(C₃-C₆-alkyl)—C(═O)N(R₇)(R₈);
4-7-membered heterocycloalkyl, wherein said 4-7-membered heterocycloalkyl is optionally substituted with one or two substituents which is independently selected from the group consisting of C₁-C₃ alkyl, 5- to 6-membered heteroaryl, —C(═O)O(C₁-C₄ alkyl), —C(═O)(C₁-C₃-alkyl), —C(═O)(C₃-C₆-cycloalkyl), —S(═O)₂(C₁-C₆-alkyl) and oxo (═O);
a phenyl which is optionally substituted with one, two, three, four or five substituents, wherein each substituent is independently selected from the group consisting of halogen, C₁-C₆ alkyl, C₃-C₈ cycloalkyl, C₁-C₆ haloalkyl, C₂-C₆-alkenyl, C₂-C₆ alkynyl, aryl, -(C₁-C₆ alkyl)-aryl, -aryl-(C₁-C₆ alkyl), hydroxyl, cyano, C₁-C₆ hydroxyalkyl, C₁-C₄ alkoxy, -O(C₂-C₆ alkenyl), C₁-C₆ haloalkoxy, C₃-C₈ cycloalkoxy, aryl, —O—aryl, cyano, —C(═O)OR₆, —C(═O)N(R₇)(R₈), —N(R₇)(R₈), -(C₁-C₆ alkyl)-N(R₇)(R₈), -(C₁-C₆-alkyl)—C(═O)OR₆, -(C₁-C₆ alkyl)—C(═O)N(R₇)(R₈), —O—C(═O)—(C₁-C₆ alkyl), —SH, —S—(C₁-C₆ alkyl), —S—(C₂-C₆ alkenyl), —S(═O)₂N(R₇)(R₈), —S(═O)₂(C₁-C₆ alkyl), —S(═O)₂—(C₂-C₆ alkenyl), —S(═O)(═NR₁₁)(C₁-C₃ alkyl), —N(O)₂, —P(═O)(C₁-C₃ alkyl)₂;
bicyclic aryl and 5-10 membered heteroaryl, wherein said bicyclic aryl and said 5-10 membered heteroaryl are optionally substituted with one, two or three substituents, wherein each substituent is independently halogen atom or a group selected from C₁-C₆ alkyl, C₃-C₈ cycloalkyl, C₁-C₆ haloalkyl, C₂-C₆ alkenyl, C₂-C₆ alkynyl, aryl, -(C₁-C₆ alkyl)-aryl, -aryl-(C₁-C₆ alkyl), hydroxy, cyano, C₁-C₆ hydroxyalkyl, C₁-C₄ alkoxy, -O(C₂-C₆ alkenyl), C₁-C₆ haloalkoxy, C₃-C₈ cycloalkoxy, aryl, —O—aryl, cyano, —C(═O)OR₆, —C(═O)N(R₇)(R₈), —N(R₇)(R₈), -(C₁-C₆ alkyl)-N(R₇)(R₈), -(C₁-C₆ alkyl)—C(═O)OR₆, -(C₁-C₆ alkyl)—C(═O)N(R₇)(R₈), —O—C(═O)—(C₁-C₆ alkyl), —S—(C₁-C₆ alkyl), —S—(C₂-C₆ alkenyl), —S(═O)₂N(R₇)(R₈), —S(═O)₂(C₁-C₆ alkyl), —S(═O)₂—(C₂-C₆ alkenyl), —S(═O)(═NR₁₁)(C₁-C₃ alkyl), —N(O)₂, —P(═O)(C₁-C₃ alkyl)₂;
R₂ is selected from the group consisting of hydrogen, D, CN and halogen;
R₃ is selected from the group consisting of halogen, C₁-C₆ alkyl, C₃-C₈ cycloalkyl, C₁-C₆ haloalkyl, C₁-C₆ hydroxyalkyl, (C₁-C₃ alkoxy)-(C₁-C₆ alkyl), C₂-C₆ alkenyl, C₂-C₆ alkynyl, C₁-C₆ alkoxy, C₁-C₆ alkylsulfanyl, (C₁-C₆ alkyl)-N(R₇)(R₈), —N(R₇)(R₈), —C(═O)OR₆, —C(═O)N(R₇)(R₈), and S(═O)(═NR₁₁)(C₁-C₃ alkyl);
R₄ is hydrogen or selected from the group consisting of C₁-C₆ alkyl, C₂-C₆ alkylenyl, C₃-C₈ cycloalkyl, C₂-C₆ haloalkyl, C₂-C₆ hydroxyalkyl and (C₂-C₆ alkyl)-N(R₇)(R₈), wherein said C₁-C₆ alkyl is optionally substituted with a group selected from C₃-C₈ cycloalkyl and phenyl, wherein said phenyl is optionally substituted with one, two or three substituents, with each substituent independently selected from the group consisting of halogen, C₁-C₃ alkyl, C₁-C₄ haloalkyl, C₁-C₃ alkoxy and hydroxyl;
R₅ is selected from the group consisting of C₁-C₆ alkyl, C₃-C₈ cycloalkyl, C₁-C₆ haloalkyl, C₁-C₆ hydroxyalkyl, C₂-C₆ alkenyl, C₂-C₆ alkynyl, C₄-C₈ cycloalkenyl, (C₁-C₆ alkyl)-N(R₇)(R₈), -(C₁-C₆ alkyl)-(4- to 7-membered nitrogen containing heterocycloalkyl) and phenyl, wherein said C₁-C₆ alkyl is optionally substituted with C₃-C₈ cycloalkyl or NR₇R₈, said 4- to 7-membered nitrogen containing heterocycloalkyl is optionally substituted with a C₁-C₃ alkyl which is connected to the 4- to 7-membered nitrogen containing heterocycloalkyl via carbon atom of the heterocycloalkyl, and said phenyl is substituted with one, two, three or four substituents, and each substituent is selected from the group consisting of halogen, C₁-C₃ alkyl, C₁-C₄ haloalkyl, C₁-C₃ alkoxy and hydroxyl;
R₆ is hydrogen or selected from the group consisting of C₁-C₆ alkyl and benzyl;
R₇ and R₈ are independently hydrogen or selected from the group consisting of C₁-C₆ alkyl, C₂-C₆ hydroxyalkyl, (C₂-C₆ alkyl)-N(R₉)(R₁₀), and C₃-C₆ cycloalkyl, or R₇ and R₈ together with the nitrogen to which they are attached represent a nitrogen containing 4- to 7-membered heterocycloalkyl, wherein said 4- to 7-membered nitrogen containing heterocycloalkyl is optionally substituted with C₁-C₃ alkyl, —S(═O)₂(C₁-C₃ alkyl) and —C(═O)O(C₁-C₄ alkyl);
R₉ and R₁₀ are independently hydrogen or C₁-C₃ alkyl, or R₉ and R₁₀ together with the nitrogen to which they are attached represent a nitrogen containing 4- to 7-membered heterocycloalkyl group;
R₁₁ is hydrogen, cyano or C(=O)(C₁-C₃ haloalkyl).

2. A compound of Formula (II), or a pharmaceutically acceptable salt, stereoisomer, prodrug, or solvate thereof, 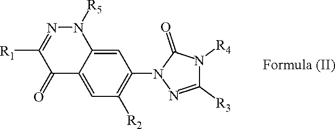
wherein:
R₁ is selected from the group consisting of
C₁-C₈ alkyl;
C₂-C₈ haloalkyl;
C₃-C₈ cycloalkyl which is optionally substituted with 1-6 halogen atoms or a group selected from hydroxyl and phenyl, wherein said phenyl substituent is optionally substituted with 1-4 halogen atoms or a group selected from C₁-C₃ alkyl, C₁-C₄ haloalkyl, C₁-C₃ alkoxy, CN and hydroxyl;
C₃-C₆ alkyl which is substituted with a monocyclic- or bicyclic heteroaryl group;
(C₂-C₆ hydroxyalkyl)-O-(C₂-C₆ alky);
(C₃-C₆ alkyl)-N(R₇)(R₈);
(C₃-C₈ cycloalkyl)-N(R₇)(R₈);
(C₃-C₆-alkyl)—C(═O)N(R₇)(R₈);
4-7-membered heterocycloalkyl, wherein said 4-7-membered heterocycloalkyl is optionally substituted with one or two substituents which is independently selected from the group consisting of C₁-C₃ alkyl, 5- to 6-membered heteroaryl, —C(═O)O(C₁-C₄ alkyl), —C(═O)(C₁-C₃-alkyl), —C(═O)(C₃-C₆-cycloalkyl), —S(═O)₂(C₁-C₆-alkyl) and oxo (═O);
a phenyl which is optionally substituted with one, two, three, four or five substituents, wherein each substituent is independently selected from the group consisting of halogen, C₁-C₆ alkyl, C₃-C₈ cycloalkyl, C₁-C₆ haloalkyl, C₂-C₆ alkenyl, C₂-C₆ alkynyl, aryl, -(C₁-C₆ alkyl)-aryl, -aryl-(C₁-C₆ alkyl), hydroxyl, cyano, C₁-C₆ hydroxyalkyl, C₁-C₄ alkoxy, -O(C₂-C₆ alkenyl), C₁-C₆ haloalkoxy, C₃-C₈ cycloalkoxy, aryl, —O—aryl,cyano, —C(═O)OR₆, —C(═O)N(R₇)(R₈), —N(R₇)(R₈), -(C₁-C₆ alkyl)-N(R₇)(R₈), -(C₁-C₆ alkyl)—C(═O)OR₆, -(C₁-C₆ alkyl)—C(═O)N(R₇)(R₈), —O—C(═O)—(C₁-C₆ alkyl), —SH, —S—(C₁-C₆ alkyl), —S—(C₂-C₆ alkenyl), —S(═O)₂N(R₇)(R₈), —S(═O)₂(C₁-C₆ alkyl), —S(═O)₂—(C₂-C₆ alkenyl), —S(═O)(═NR₁₁)(C₁-C₃ alkyl), —N(O)₂, —P(═O)(C₁-C₃ alkyl)₂;
bicyclic aryl and 5-10 membered heteroaryl, wherein said bicyclic aryl and said 5-10 membered heteroaryl are optionally substituted with one, two or three substituents, wherein each substituent is independently halogen atom or a group selected from C₁-C₆ alkyl, C₃-C₈ cycloalkyl, C₁-C₆ haloalkyl, C₂-C₆ alkenyl, C₂-C₆ alkynyl, aryl, -(C₁-C₆ alkyl)-aryl, -aryl-(C₁-C₆ alkyl), hydroxy, cyano, C₁-C₆ hydroxyalkyl, C₁-C₄ alkoxy, -O(C₂-C₆ alkenyl), C₁-C₆ haloalkoxy, C₃-C₈ cycloalkoxy, aryl, —O—aryl, cyano, —C(═O)OR₆, —C(═O)N(R₇)(R₈), —N(R₇)(R₈), -(C₁-C₆ alkyl)-N(R₇)(R₈), -(C₁-C₆ alkyl)—C(═O)OR₆, -(C₁-C₆ alkyl)—C(═O)N(R₇)(R₈), —O—C(═O)—(C₁-C₆ alkyl), —S—(C₁-C₆ alkyl), —S—(C₂-C₆ alkenyl), —S(═O)₂N(R₇)(R₈), —S(═O)₂(C₁-C₆ alkyl), —S(═O)₂—(C₂-C₆ alkenyl), —S(═O)(═NR₁₁)(C₁-C₃ alkyl), —N(O)₂, —P(═O)(C₁-C₃-alkyl)₂;
R₂ is selected from the group consisting of hydrogen, D, CN and halogen;
R₃ is selected from the group consisting of halogen, C₁-C₆ alkyl, C₃-C₈ cycloalkyl, C₁-C₆ haloalkyl, C₁-C₆ hydroxyalkyl, (C₁-C₃ alkoxy)-(C₁-C₆ alkyl), C₂-C₆ alkenyl, C₂-C₆ alkynyl, C₁-C₆ alkoxy, C₁-C₆ alkylsulfanyl, (C₁-C₆ alkyl)-N(R₇)(R₈), —N(R₇)(R₈), —C(═O)OR₆, —C(═O)N(R₇)(R₈), and S(═O)(═NR₁₁)(C₁-C₃ alkyl);
R₄ is hydrogen or selected from the group consisting of C₁-C₆ alkyl, C₂-C₆ alkylenyl, C₃-C₈ cycloalkyl, C₂-C₆ haloalkyl, C₂-C₆ hydroxyalkyl and (C₂-C₆ alkyl)-N(R₇)(R₈), wherein said C₁-C₆ alkyl is optionally substituted with a group selected from C₃-C₈ cycloalkyl and phenyl, wherein said phenyl is optionally substituted with one, two or three substituents, with each substituent independently selected from the group consisting of halogen, C₁-C₃ alkyl, C₁-C₄ haloalkyl, C₁-C₃ alkoxy and hydroxyl;
R₅ is selected from the group consisting of C₁-C₆ alkyl, C₃-C₈ cycloalkyl, C₁-C₆ haloalkyl, C₁-C₆ hydroxyalkyl, C₂-C₆ alkenyl, C₂-C₆ alkynyl, C₄-C₈ cycloalkenyl, (C₁-C₆ alkyl)-N(R₇)(R₈), -(C₁-C₆ alkyl)-(4- to 7-membered nitrogen containing heterocycloalkyl) and phenyl, wherein said C₁-C₆ alkyl is optionally substituted with C₃-C₈ cycloalkyl or NR₇R₈, said 4- to 7-membered nitrogen containing heterocycloalkyl is optionally substituted with a C₁-C₃ alkyl which is connected to the 4- to 7-membered nitrogen containing heterocycloalkyl via carbon atom of the heterocycloalkyl, and said phenyl is substituted with 1-4 halogen atoms or C₁-C₃ alkyl, C₁-C₄ haloalkyl, C₁-C₃ alkoxy and hydroxyl;
R₆ is hydrogen or selected from the group consisting of C₁-C₆ alkyl and benzyl;
R₇ and R₈ are independently hydrogen or selected from the group consisting of C₁-C₆ alkyl, C₂-C₆ hydroxyalkyl, (C₂-C₆ alkyl)-N(R₉)(R₁₀), and C₃-C₆ cycloalkyl, or R₇ and R₈ together with the nitrogen to which they are attached represent a nitrogen containing 4- to 7-membered heterocycloalkyl, wherein said 4- to 7-membered nitrogen containing heterocycloalkyl is optionally substituted with C₁-C₃ alkyl, —S(═O)₂(C₁-C₃ alkyl) and —C(═O)O(C₁-C₄ alkyl);
R₉ and R₁₀ are independently hydrogen or C₁-C₃ alkyl, or R₉ and R₁₀ together with the nitrogen to which they are attached represent a nitrogen containing 4- to 7-membered heterocycloalkyl group;
R₁₁ is hydrogen, cyano or C(=O)(C₁-C₃ haloalkyl).

3. The compound of claim 1, or a pharmaceutically acceptable salt, stereoisomer, prodrug, or solvate thereof, wherein R₁ is a phenyl which is optionally substituted with one, two or three substituents, wherein each substituent is independently selected from the group consisting of F, Cl, C₁-C₆ alkyl, C₃-C₆ cycloalkyl, C₁-C₃ haloalkyl, C₂-C₄ alkenyl, C₂-C₄ alkynyl, aryl, -(C₁-C₆ alkyl)-aryl, -aryl-(C₁-C₆ alkyl), hydroxyl, cyano, C₁-C₄ hydroxyalkyl, C₁-C₄ alkoxy, -O(C₂-C₄ alkenyl), C₁-C₄ haloalkoxy, C₃-C₆ cycloalkoxy, aryl, —O—aryl, cyano, —O—C(═O)—(C₁-C₆ alkyl), —SH, —S—(C₁-C₆ alkyl), —S—(C₂-C₆ alkenyl), —S(═O)₂(C₁-C₆ alkyl), —S(═O)₂—(C₂-C₆ alkenyl), —N(O)₂, —P(═O)(C₁-C₃ alkyl)₂.

4. The compound of claim 1, or a pharmaceutically acceptable salt, stereoisomer, prodrug, or solvate thereof, wherein R₁ is C₅-C₈ alkyl, C₂-C₆ haloalkyl, or C₄-C₇ cycloalkyl which is optionally partially unsaturated and is optionally substituted with one or two substituents, wherein each substituent is independently selected from F, Cl, phenyl and hydroxyl, and said phenyl substituent is optionally substituted with one, two or three substituents which selected from F, Cl, C₁-C₃ alkyl, C₁-C₄ haloalkyl, C₁-C₃ alkoxy and hydroxy.

5. The compound of claim 1, or a pharmaceutically acceptable salt, stereoisomer, prodrug, or solvate thereof, wherein R₁ is C₁-C₆ alkyl which is substituted with C₃-C₆ cycloalkyl, C₂-C₆ alkyl which is substituted with cyano, hydroxyl, phenyl or C3-C8 heterocycloalkyl, 4-7-membered heterocycloalkyl which is optionally substituted with one or two substituents, wherein each substituent is independently selected from C₁-C₃ alkyl, 5- to 6-membered heteroaryl, —C(═O)O(C₁-C₄-alkyl), —C(═O)(C₁-C₃-alkyl), and —C(═O)(C₃-C₆-cycloalkyl).

6. The compound of claim 1, or a pharmaceutically acceptable salt, stereoisomer, prodrug, or solvate thereof, wherein R₂ is H or F or CN.

7. The compound of claim 1, or a pharmaceutically acceptable salt, stereoisomer, prodrug, or solvate thereof, wherein R₃ is selected from the group consisting of C₁-C₃ alkyl, C₃-C₆ cycloalkyl, C₁-C₃ hydroxyalkyl, (C₁-C₃ alkoxy)-(C₁-C₆ alkyl), C₁-C₆ alkoxy, —C(═O)OH, and —S(═O)(═NH)(C₁-C₃ alky).

8. The compound of claim 1, or a pharmaceutically acceptable salt, stereoisomer, prodrug, or solvate thereof, wherein R₄ is selected from the group consisting of C₁-C₆ alkyl, C₂-C₆ alkylenyl, C₃-C₆ cycloalkyl, C₂-C₆ haloalkyl and C₂-C₆ hydroxyalkyl.

9. The compound of claim 1, or a pharmaceutically acceptable salt, stereoisomer, prodrug, or solvate thereof, wherein R₅ is selected from the group consisting of C₁-C₆ alkyl, C₃-C₈ cycloalkyl, C₁-C₆ haloalkyl, C₁-C₆ hydroxyalkyl, C₂-C₆ alkenyl, C₂-C₆ alkynyl, C₄-C₈ cycloalkenyl and phenyl; wherein said phenyl is substituted with one, two, three or four substituents, each substituent is selected from the group consisting of F, Cl, C₁-C₃ alkyl, C₁-C₄ haloalkyl, C₁-C₃ alkoxy and hydroxyl.

10. The compound of claim 1, or a pharmaceutically acceptable salt, stereoisomer, prodrug, or solvate thereof, wherein the compound is selected from the group consisting of:
3-(2-chloro-6-fluorophenyl)-7-(4-ethyl-3-(hydroxymethyl)-5-oxo-4,5-dihydro-1H-1,2,4-triazol-1-yl)-6-fluoro-1-(1,1,1-trifluoropropan-2-yl)cinnolin-4(1H)-one;
3-(2-chloro-6-fluorophenyl)-7-(4-ethyl-3-(hydroxymethyl)-5-oxo-4,5-dihydro-1H-1,2,4-triazol-1-yl)-6-fluoro-1-isopropylcinnolin-4(1H)-one;
3-(2-chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one;
2-(2-chloro-6-fluorophenyl)-6-(4-ethyl-3-(hydroxymethyl)-5-oxo-4,5-dihydro-1H-1,2,4-triazol-1-yl)-7-fluoro-4-isopropylphthalazin-1(2H)-one;
3-(2-chloro-6-fluorophenyl)-1-(1-cyclopropylethyl)-7-(4-ethyl-3-(hydroxymethyl)-5-oxo-4,5-dihydro-1H-1,2,4-triazol-1-yl)-6-fluorocinnolin-4(1H)-one;
3-(2-chloro-6-fluorophenyl)-7-(4-ethyl-3-(1-hydroxyethyl)-5-oxo-4,5-dihydro-1H-1,2,4-triazol-1-yl)-6-fluoro-1-isopropylcinnolin-4(1H)-one;
1-(3-(2-chloro-6-fluorophenyl)-6-fluoro-1-isopropyl-4-oxo-1,4-dihydrocinnolin-7-yl)-4-ethyl-5-oxo-4,5-dihydro-1H-1,2,4-triazole-3-carboxamide;
3-(2,6-difluorophenyl)-7-(4-ethyl-3-(hydroxymethyl)-5-oxo-4,5-dihydro-1H-1,2,4-triazol-1-yl)-6-fluoro-1-isopropylcinnolin-4(1H)-one;
3-(2-chloro-6-fluorophenyl)-6-fluoro-7-(3-(hydroxymethyl)-5-oxo-4-(2,2,2-trifluoroethyl)-4,5-dihydro-1H-1,2,4-triazol-1-yl)-1-isopropylcinnolin-4(1H)-one;
7-(4-ethyl-3-(hydroxymethyl)-5-oxo-4,5-dihydro-1H-1,2,4-triazol-1-yl)-6-fluoro-3-(2-fluoro-6-(trifluoromethyl)phenyl)-1-isopropylcinnolin-4(1H)-one;
3-(2-chloro-6-fluorophenyl)-7-(4-ethyl-3-(hydroxymethyl)-5-oxo-4,5-dihydro-1H-1,2,4-triazol-1-yl)-6-fluoro-1-(hexan-2-yl)cinnolin-4(1H)-one;
2-(2-chloro-6-fluorophenyl)-6-(4-ethyl-3-(hydroxymethyl)-5-oxo-4,5-dihydro-1H-1,2,4-triazol-1-yl)-7-fluoro-4-(1,1,1-trifluoropropan-2-yl)phthalazin-1(2H)-one;
2-(2-chloro-6-fluorophenyl)-4-(1-cyclopropylethyl)-6-(4-ethyl-3-(hydroxymethyl)-5-oxo-4,5-dihydro-1H-1,2,4-triazol-1-yl)-7-fluorophthalazin-1(2H)-one;
2-(2-chloro-6-fluorophenyl)-6-(4-ethyl-3-(hydroxymethyl)-5-oxo-4,5-dihydro-1H-1,2,4-triazol-1-yl)-7-fluoro-4-(pentan-2-yl)phthalazin-1(2H)-one;
6-(4-ethyl-3-(hydroxymethyl)-5-oxo-4,5-dihydro-1H-1,2,4-triazol-1-yl)-7-fluoro-2-(2-fluoro-6-methylphenyl)-4-(1, 1, 1-trifluoropropan-2-yl)phthalazin-1 (2H)-one;
1-(2-(2-chloro-6-fluorophenyl)-7-fluoro-1-oxo-4-(1,1,1-trifluoropropan-2-yl)-1,2-dihydrophthalazin-6-yl)-4-ethyl-5-oxo-4,5-dihydro-1H-1,2,4-triazole-3-carboxamide;
2-(2-chloro-6-fluorophenyl)-4-(1-cyclopropylethyl)-6-(4-ethyl-3-(1-hydroxyethyl)-5-oxo-4,5-dihydro-1H-1,2,4-triazol-1-yl)-7-fluorophthalazin-1(2H)-one;
7-fluoro-6-(3-(hydroxymethyl)-4-methyl-5-oxo-4,5-dihydro-1H-1,2,4-triazol-1-yl)-4-isopropyl-2-(o-tolyl)phthalazin-1(2H)-one;
2-(2-chloro-6-fluorophenyl)-4-(1-cyclobutylethyl)-6-(4-cyclopropyl-3-(hydroxymethyl)-5-oxo-4,5-dihydro-1H-1,2,4-triazol-1-yl)-7-fluorophthalazin-1(2H)-one;
2-(2-chloro-6-fluorophenyl)-6-(3-(hydroxymethyl)-4-isopropyl-5-oxo-4,5-dihydro-1H-1,2,4-triazol-1-yl)-4-(1,1,1-trifluoropropan-2-yl)phthalazin-1(2H)-one;
2-(2-chloro-6-fluorophenyl)-6-(4-ethyl-3-(hydroxymethyl)-5-oxo-4,5-dihydro-1H-1,2,4-triazol-1-yl)-7-fluoro-4-isopropylisoquinolin-1(2H)-one;
7-(4-ethyl-3-(hydroxymethyl)-5-oxo-4,5-dihydro-1H-1,2,4-triazol-1-yl)-6-fluoro-1-isopropyl-3-(o-tolyl)cinnolin-4(1H)-one.

11. A pharmaceutical composition comprising:
(i) a compound of claim 1, or a pharmaceutically acceptable salt, stereoisomer, prodrug, or solvate thereof; and
(ii) one or more pharmaceutically acceptable carriers.

12. A method for treating or preventing DHODH-mediated diseases, conditions or disorders, comprising:
administering a subject in need a therapeutically effective amount of a compound of claim 1, or a pharmaceutically acceptable salt, stereoisomer, prodrug, or solvate thereof.

13. The method of claim 12, wherein DHODH-mediated diseases, conditions or disorders are virus infection, cancer or inflammatory disorders.

14. (canceled)

15. A method for treating or preventing DHODH-mediated diseases, conditions or disorders, comprising:
administering a subject in need a therapeutically effective amount of a pharmaceutical composition of claim 11.
